SEQUENCE LISTING (34 sequences)

SEQ ID NO: 1 - protein, 113 aa, Brugia malayi
MEEKVVELTHNWSAEQWDWPLQHNDEVIKVTNTNDKFEVGLDASFFTPKEIEVKVAGDNLVIHCRHESRAEHYGEIKREISRTYKLPSDVDTKTLTSNLTKRGHLVIAAKKKA

SEQ ID NO: 2 - protein, 129 aa, Brugia malayi
MGNKLLIAFGLVILFVTLPCVSESDEEFDDSAADDTDDSEAGGGSEGGDEYVTKGEFVETDGKKKECSSHEACYDQREPQAWCRLSENQAWTDRGCFCEDKLHSCVIERTNNGKLEYSYCAPEAGWQCA

SEQ ID NO: 3 - protein, 199 aa, Brugia malayi
MTLAGSKAFIGQPAPNFKTTAVVNGDFKEISLGQFKGKYVVLLFYPLDFTFVCPTEIIAFSDRIAEFKQLDVAVMACSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVCPANWHPGSETIKPGVKESKAYFEKH

SEQ ID NO: 4 - protein, 89 aa, Brugia malayi
MILEVASIVLIIAGKDQFKNALYNLLSKTGESEDEMQHFKPIEDLFQCCGPTNETMVRYIENGLCEDELRNKPNCFAVISDHFDSSQKD

SEQ ID NO: 5 - protein, 113 aa, Artificial sequence
  Synthetic polypeptide
MEEKVVELTHNWSAEQWDWPLQHNDEVIKVTNTNDKFEVGLDASFFTPKEIEVKVAGDNLVIHSRHESRAEHYGEIKREISRTYKLPSDVDTKTLTSNLTKRGHLVIAAKKKA

SEQ ID NO: 6 - protein, 129 aa, Artificial sequence
  Synthetic polypeptide
MGNKLLIAFGLVILFVTLPSVSESDEEFDDSAADDTDDSEAGGGSEGGDEYVTKGEFVETDGKKKESSSHEASYDQREPQAWSRLSENQAWTDRGSFSEDKLHSSVIERTNNGKLEYSYSAPEAGWQSA

SEQ ID NO: 7 - protein, 199 aa, Artificial sequence
  Synthetic polypeptide
MTLAGSKAFIGQPAPNFKTTAVVNGDFKEISLGQFKGKYVVLLFYPLDFTFVSPTEIIAFSDRIAEFKQLDVAVMASSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVSPANWHPGSETIKPGVKESKAYFEKH

SEQ ID NO: 8 - protein, 89 aa, Artificial sequence
  Synthetic polypeptide
MILEVASIVLIIAGKDQFKNALYNLLSKTGESEDEMQHFKPIEDLFQSSGPTNETMVRYIENGLSEDELRNKPNSFAVISDHFDSSQKD

SEQ ID NO: 9 - protein, 524 aa, Artificial sequence
  Synthetic polypeptide
MHHHHHHEEKVVELTHNWSAEQWDWPLQHNDEVIKVTNTNDKFEVGLDASFFTPKEIEVKVAGDNLVIHCRHESRAEHYGEIKREISRTYKLPSDVDTKTLTSNLTKRGHLVIAAKKKAMNKLLIAFGLVILFVTLPCVSESDEEFDDSAADDTDDSEAGGGSEGGDEYVTKGEFVETDGKKKECSSHEACYDQREPQAWCRLSENQAWTDRGCFCEDKLHSCVIERTNNGKLEYSYCAPEAGWQCAMTLAGSKAFIGQPAPNFKTTAVVNGDFKEISLGQFKGKYVVLLFYPLDFTFVCPTEIIAFSDRIAEFKQLDVAVMACSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVCPANWHPGSETIKPGVKESKAYFEKHIAGKDQFKNALYNLLSKTGESEDEMQHFKPIEDLFQCCGPTNETMVRYIENGLCEDELRNKPNCFAVISDHFDSSQKD

SEQ ID NO: 10 - protein, 518 aa, Artificial sequence
  Synthetic polypeptide
MEEKVVELTHNWSAEQWDWPLQHNDEVIKVTNTNDKFEVGLDASFFTPKEIEVKVAGDNLVIHSRHESRAEHYGEIKREISRTYKLPSDVDTKTLTSNLTKRGHLVIAAKKKAMNKLLIAFGLVILFVTLPSVSESDEEFDDSAADDTDDSEAGGGSEGGDEYVTKGEFVETDGKKKESSSHEASYDQREPQAWSRLSENQAWTDRGSFSEDKLHSSVIERTNNGKLEYSYSAPEAGWQSAMTLAGSKAFIGQPAPNFKTTAVVNGDFKEISLGQFKGKYVVLLFYPLDFTFVSPTEIIAFSDRIAEFKQLDVAVMASSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVSPANWHPGSETIKPGVKESKAYFEKHIAGKDQFKNALYNLLSKTGESEDEMQHFKPIEDLFQSSGPTNETMVRYIENGLSEDELRNKPNSFAVISDHFDSSQKD

SEQ ID NO: 11 - protein, 554 aa, Artificial sequence
  Synthetic polypeptide
MEEKVVELTHNWSAEQWDWPLQHNDEVIKVTNTNDKFEVGLDASFFTPKEIEVKVAGDNLVIHCRHESRAEHYGEIKREISRTYKLPSDVDTKTLTSNLTKRGHLVIAAKKKAGGGSGGGSGGGSMNKLLIAFGLVILFVTLPCVSESDEEFDDSAADDTDDSEAGGGSEGGDEYVTKGEFVETDGKKKECSSHEACYDQREPQAWCRLSENQAWTDRGCFCEDKLHSCVIERTNNGKLEYSYCAPEAGWQCAGGGSGGGSGGGSMTLAGSKAFIGQPAPNFKTTAVVNGDFKEISLGQFKGKYVVLLFYPLDFTFVCPTEIIAFSDRIAEFKQLDVAVMACSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVCPANWHPGSETIKPGVKESKAYFEKHGGGSGGGSGGGSIAGKDQFKNALYNLLSKTGESEDEMQHFKPIEDLFQCCGPTNETMVRYIENGLCEDELRNKPNCFAVISDHFDSSQKD

SEQ ID NO: 12 - protein, 554 aa, Artificial sequence
  Synthetic polypeptide
MEEKVVELTHNWSAEQWDWPLQHNDEVIKVTNTNDKFEVGLDASFFTPKEIEVKVAGDNLVIHSRHESRAEHYGEIKREISRTYKLPSDVDTKTLTSNLTKRGHLVIAAKKKAGGGSGGGSGGGSMNKLLIAFGLVILFVTLPSVSESDEEFDDSAADDTDDSEAGGGSEGGDEYVTKGEFVETDGKKKESSSHEASYDQREPQAWSRLSENQAWTDRGSFSEDKLHSSVIERTNNGKLEYSYSAPEAGWQSAGGGSGGGSGGGSMTLAGSKAFIGQPAPNFKTTAVVNGDFKEISLGQFKGKYVVLLFYPLDFTFVSPTEIIAFSDRIAEFKQLDVAVMASSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVSPANWHPGSETIKPGVKESKAYFEKHGGGSGGGSGGGSIAGKDQFKNALYNLLSKTGESEDEMQHFKPIEDLFQSSGPTNETMVRYIENGLSEDELRNKPNSFAVISDHFDSSQKD

SEQ ID NO: 13 - protein, 524 aa, Artificial sequence
  Synthetic polypeptide
MHHHHHHEEKVVELTHNWSAEQWDWPLQHNDEVIKVTNTNDKFEVGLDASFFTPKEIEVKVAGDNLVIHSRHESRAEHYGEIKREISRTYKLPSDVDTKTLTSNLTKRGHLVIAAKKKAMNKLLIAFGLVILFVTLPSVSESDEEFDDSAADDTDDSEAGGGSEGGDEYVTKGEFVETDGKKKESSSHEASYDQREPQAWSRLSENQAWTDRGSFSEDKLHSSVIERTNNGKLEYSYSAPEAGWQSAMTLAGSKAFIGQPAPNFKTTAVVNGDFKEISLGQFKGKYVVLLFYPLDFTFVSPTEIIAFSDRIAEFKQLDVAVMASSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVSPANWHPGSETIKPGVKESKAYFEKHIAGKDQFKNALYNLLSKTGESEDEMQHFKPIEDLFQSSGPTNETMVRYIENGLSEDELRNKPNSFAVISDHFDSSQKD

SEQ ID NO: 14 - protein, 560 aa, Artificial sequence
  Synthetic polypeptide
MHHHHHHEEKVVELTHNWSAEQWDWPLQHNDEVIKVTNTNDKFEVGLDASFFTPKEIEVKVAGDNLVIHCRHESRAEHYGEIKREISRTYKLPSDVDTKTLTSNLTKRGHLVIAAKKKAGGGSGGGSGGGSMNKLLIAFGLVILFVTLPCVSESDEEFDDSAADDTDDSEAGGGSEGGDEYVTKGEFVETDGKKKECSSHEACYDQREPQAWCRLSENQAWTDRGCFCEDKLHSCVIERTNNGKLEYSYCAPEAGWQCAGGGSGGGSGGGSMTLAGSKAFIGQPAPNFKTTAVVNGDFKEISLGQFKGKYVVLLFYPLDFTFVCPTEIIAFSDRIAEFKQLDVAVMACSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVCPANWHPGSETIKPGVKESKAYFEKHGGGSGGGSGGGSIAGKDQFKNALYNLLSKTGESEDEMQHFKPIEDLFQCCGPTNETMVRYIENGLCEDELRNKPNCFAVISDHFDSSQKD

SEQ ID NO: 15 - protein, 560 aa, Artificial sequence
  Synthetic polypeptide
MHHHHHHEEKVVELTHNWSAEQWDWPLQHNDEVIKVTNTNDKFEVGLDASFFTPKEIEVKVAGDNLVIHSRHESRAEHYGEIKREISRTYKLPSDVDTKTLTSNLTKRGHLVIAAKKKAGGGSGGGSGGGSMNKLLIAFGLVILFVTLPSVSESDEEFDDSAADDTDDSEAGGGSEGGDEYVTKGEFVETDGKKKESSSHEASYDQREPQAWSRLSENQAWTDRGSFSEDKLHSSVIERTNNGKLEYSYSAPEAGWQCAGGGSGGGSGGGSMTLAGSKAFIGQPAPNFKTTAVVNGDFKEISLGQFKGKYVVLLFYPLDFTFVSPTEIIAFSDRIAEFKQLDVAVMASSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVSPANWHPGSETIKPGVKESKAYFEKHGGGSGGGSGGGSIAGKDQFKNALYNLLSKTGESEDEMQHFKPIEDLFQSSGPTNETMVRYIENGLSEDELRNKPNSFAVISDHFDSSQKD

SEQ ID NO: 16 - DNA, 1575 bp, Artificial sequence
  Synthetic polynucleotide
atgcatcaccatcaccatcacgaagaaaaggtagtggaactaacgcacaactggagtgcggagcagtgggattggcccttacagcacaacgatgaggtgattaaagtaaccaacacgaatgataagtttgaagtcggtttggatgcatcattcttcacgccgaaggaaatcgaggtaaaagttgctggcgataatctggtcattcactgcagacatgaatcacgtgccgagcattatggagaaattaaacgtgaaattagtcgtacctataagcttccgtcggatgtagatacgaagactctgacatcaaatttgaccaagcgaggacatttggtcatcgctgccaaaaagaaagcaatgaataaacttttaatagcattcggtttggtaattctttttgtgacactcccgtgtgtatcagaatcagacgaagagttcgatgactccgcagccgatgacaccgacgacagcgaggccggaggtggtagtgaaggaggtgatgaatatgtaaccaaaggggaatttgttgaaactgatggcaaaaagaaagagtgctcttcgcacgaagcttgctacgatcaacgtgaaccacaagcgtggtgcagactgagcgagaatcaggcatggactgacagaggctgcttctgcgaagataagttgcattcgtgcgtcatcgaaagaacgaacaatggtaaattggagtattcgtactgtgcacctgaagcaggttggcaatgcgcaatgacacttgctggaagtaaagcatttattggtcaaccagcacctaattttaaaacaacagctgttgtgaacggtgatttcaaggaaatttcacttggtcaatttaagggaaaatatgttgttctcctcttttatcctcttgatttcacttttgtttgtccaacggaaataattgcattttctgatcgtattgcggaatttaaacaattagatgtagctgttatggcatgttcaacagattcacacttttcacatcttgcatgggtaaatactgatcgaaaaatgggtggacttggtcaaatgaatataccaattttggcttatacaaatcatgtaataagtcgagcatatggtgtacttaaagaggatgatggaattgcttatcgtggattattcattattgatccaaaaggaattttggggcaaatcacaattaacgatcttccagtagggcgttctgtagacgaaactttacgtttaattcaagcttttcagtttgttgacaagcatggtgaagtatgtcctgctaattggcatccaggatctgaaacgattaagcctggtgtgaaagaaagtaaagcatattttgagaaacatattgctggcaaggatcaatttaaaaatgctttatataatttactatcaaaaactggcgaatcagaagatgaaatgcaacattttaaacctatcgaagatttattccaatgttgtggtccaacaaatgaaacaatggttcgatacatcgagaatggcttatgtgaggatgaattaagaaataaaccgaattgtttcgcagtaatatccgatcattttgattcatctcaaaaagattaa

SEQ ID NO: 17 - DNA, 1556 bp, Artificial sequence
  Synthetic polynucleotide
atggaagaaaaagtagtggaattaacgcacaactggagtgccgaacaatgggattggcctttacaacataatgacgaggtgattaaagtaacaaatactaatgataaatttgaagtaggtctggacgcttccttttttacccccaaggaaattgaagtaaaagtcgccggagacaacttagtaattcatagccgccatgagagtcgcgcagagcattacggcgaaatcaaacgcgagatttcacgcacctacaagctgccaagtgatgtagacactaagactttaacatcaaacctgactaagcgcgggcatttggtgattgcagctaagaaaaaagctatgaataagctgttaatcgctttcggcttagtgatcctgtttgtgacattaccttctgtttccgagtccgacgaagaatttgacgattctgccgccgatgacactgacgactccgaagcaggcggcggatcggagggcggtgacgaatacgttactaagggggagtttgtagagacagatggtaaaaagaaagagtctagtagtcacgaagccagctatgaccagcgtgaaccccaggcttggtctcgcctgtcggagaatcaggcatggactgatcgcgggtccttctctgaagataaattgcactccagcgtaattgaacgtacaaacaacggcaagctggaatactcgtattccgctcccgaagctggatggcagagcgccatgacgttagctggttccaaagcgttcattggtcagccagcaccaaatttcaagaccacagcggtcgttaatggcgacttcaaagagatttcacttgggcaattcaaagggaagtacgtggtattgcttttttatccgctggatttcacgttcgtgagccctaccgagattattgctttctctgaccgtatcgcagaatttaagcagttagacgtggctgtaatggcctctagcacagactcacacttcagtcatctggcttgggtcaacacggatcgtaagatgggcgggctgggacaaatgaacatccctatccttgcctatactaatcacgtaatctcccgtgcttatggtgtgttaaaagaggatgacggcattgcctaccgtggattgttcattattgatccgaagggtattttagggcaaatcacaattaacgaccttcctgttggccgttccgtggacgaaacactgcgcctgattcaggcgttccagtttgtcgataagcatggcgaggtgtctccagcaaattggcatcccggttcagaaacgatcaaacccggtgttaaggaatctaaagcctattttgagaaacacattgcaggcaaagaccagttcaaaaatgcactgtataacttgttatctaagacaggggaaagcgaagatgaaatgcagcatttcaaacccattgaggacctttttcagtccagcggacctacaaatgagactatggtacgttatatcgaaaacggtctttcgaggacgaactgcgtaataagcctaacagctttgcggtgattagcgatcactttgattcatcacaaaaggattaa

SEQ ID NO: 18 - DNA, 1664 bp, Artificial sequence
  Synthetic polynucleotide
atggaagaaaaagtagtggaattaacgcacaactggagtgccgaacaatgggattggcctttacaacataatgacgaggtgattaaagtaacaaatactaatgataaatttgaagtaggtctggacgcttccttttttacccccaaggaaattgaagtaaaagtcgccggagacaacttagtaattcattgccgccatgagagtcgcgcagagcattacggcgaaatcaaacgcgagatttcacgcacctacaagctgccaagtgatgtagacactaagactttaacatcaaacctgactaagcgcgggcatttggtgattgcagctaagaaaaaagctggcggcggtagcggcggtggctctggcggtggttccatgaataagctgttaatcgctttcggcttagtgatcctgtttgtgacattaccttgtgtttccgagtccgacgaagaatttgacgattctgccgccgatgacactgacgactccgaagcaggcggcggatcggagggcggtgacgaatacgttactaagggggagtttgtaggacagatggtaaaaagaaagagtgtagtagtcacgaagcctgctatgaccagcgtgaaccccaggcttggtgtcgcctgtcggagaatcaggcatggactgatcgcgggtgcttctgtgaagataaattgcactcctgcgtaattgaacgtacaaacaacggcaagctggaatactcgtattgcgctcccgaagctggatggcagtgcgccggtggcggttctggtggcggctccggtggtggcagcatgacgttagctggttccaaagcgttcattggtcagccagcaccaaatttcaagaccacagcggtcgttaatggcgacttcaaagagatttcacttgggcaattcaaagggaagtacgtggtattgcttttttatccgctggatttcacgttcgtgtgccctaccgagattattgctttctctgaccgtatcgcagaatttaagcagttagacgtggctgtaatggcctgtagcacagactcacacttcagtcatctggcttgggtcaacacggatcgtaagatgggcgggctgggacaaatgaacatccctatccttgcctatactaatcacgtaatctcccgtgcttatggtgtgttaaaagaggatgacggcattgcctaccgtggattgttcattattgatccgaagggtattttagggcaaatcacaattaacgaccttcctgttggccgttccgtggacgaaacactgcgcctgattcaggcgttccagtttgtcgataagcatggcgaggtgtgtccagcaaattggcatcccggttcagaaacgatcaaacccggtgttaaggaatctaaagcctattttgagaaacacggtggtggctccggtggcggtagcggcggcggttctattgcaggcaaagaccagttcaaaaatgcactgtataacttgttatctaagacaggggaaagcgaagatgaaatgcagcatttcaaacccattgaggacctttttcagtgctgcggacctacaaatgagactatggtacgttatatcgaaaacggtctttgtgaggacgaactgcgtaataagcctaactgctttgcggtgattagcgatcactttgattcatcacaaaaggattaa

SEQ ID NO: 19 - DNA, 1665 bp, Artificial sequence
  Synthetic polynucleotide
atggaagaaaaagtagtggaattaacgcacaactggagtgccgaacaatgggattggcctttacaacataatgacgaggtgattaaagtaacaaatactaatgataaatttgaagtaggtctggacgcttccttttttacccccaaggaaattgaagtaaaagtcgccggagacaacttagtaattcatagccgccatgagagtcgcgcagagcattacggcgaaatcaaacgcgagatttcacgcacctacaagctgccaagtgatgtagacactaagactttaacatcaaacctgactaagcgcgggcatttggtgattgcagctaagaaaaaagctggcggcggtagcggcggtggctctggcggtggttccatgaataagctgttaatcgctttcggcttagtgatcctgtttgtgacattaccttctgtttccgagtccgacgaagaatttgacgattctgccgccgatgacactgacgactccgaagcaggcggcggatcggagggcggtgacgaatacgttactaagggggagtttgtagagacagatggtaaaaagaaagagtctagtagtcacgaagccagctatgaccagcgtgaaccccaggcttggtctcgcctgtcggagaatcaggcatggactgatcgcgggtccttctctgaagataaattgcactccagcgtaattgaacgtacaaacaacggcaagctggaatactcgtattccgctcccgaagctggatggcagagcgccggtggcggttctggtggcggctccggtggtggcagcatgacgttagctggttccaaagcgttcattggtcagccagcaccaaatttcaagaccacagcggtcgttaatggcgacttcaaagagatttcacttgggcaattcaaagggaagtacgtggtattgcttttttatccgctggatttcacgttcgtgagccctaccgagattattgctttctctgaccgtatcgcagaatttaagcagttagacgtggctgtaatggcctctagcacagactcacacttcagtcatctggcttgggtcaacacggatcgtaagatgggcgggctgggacaaatgaacatccctatccttgcctatactaatcacgtaatctcccgtgcttatggtgtgttaaaagaggatgacggcattgcctaccgtggattgttcattattgatccgaagggtattttagggcaaatcacaattaacgaccttcctgttggccgttccgtggacgaaacactgcgcctgattcaggcgttccagtttgtcgataagcatggcgaggtgtctccagcaaattggcatcccggttcagaaacgatcaaacccggtgttaaggaatctaaagcctattttgagaaacacggtggtggctccggtggcggtagcggcggcggttctattgcaggcaaagaccagttcaaaaatgcactgtataacttgttatctaagacaggggaaagcgaagatgaaatgcagcatttcaaacccattgaggacctttttcagtccagcggacctacaaatgagactatggtacgttatatcgaaaacggtctttctgaggacgaactgcgtaataagcctaacagctttgcggtgattagcgatcactttgattcatcacaaaaggattaa

SEQ ID NO: 20 - DNA, 1557 bp, Artificial sequence
  Synthetic polynucleotide
atggaagaaaaagtagtggaattaacgcacaactggagtgccgaacaatgggattggcctttacaacataatgacgaggtgattaaagtaacaaatactaatgataaatttgaagtaggtctggacgcttccttttttacccccaaggaaattgaagtaaaagtcgccggagacaacttagtaattcattgccgccatgagagtcgcgcagagcattacggcgaaatcaaacgcgagatttcacgcacctacaagctgccaagtgatgtagacactaagactttaacatcaaacctgactaagcgcgggcatttggtgattgcagctaagaaaaaagctatgaataagctgttaatcgctttcggcttagtgatcctgtttgtgacattaccttgtgtttccgagtccgacgaagaatttgacgattctgccgccgatgacactgacgactccgaagcaggcggcggatcggagggcggtgacgaatacgttactaagggggagtttgtagagacagatggtaaaaagaaagagtgtagtagtcacgaagcctgctatgaccagcgtgaaccccaggcttggtgtcgcctgtcggagaatcaggcatggactgatcgcgggtgcttctgtgaagataaattgcactcctgcgtaattgaacgtacaaacaacggcaagctggaatactcgtattgcgctcccgaagctggatggcagtgcgccatgacgttagctggttccaaagcgttcattggtcagccagcaccaaatttcaagaccacagcggtcgttaatggcgacttcaaagagatttcacttgggcaattcaaagggaagtacgtggtattgcttttttatccgctggatttcacgttcgtgtgccctaccgagattattgctttctctgaccgtatcgcagaatttaagcagttagacgtggctgtaatggcctgtagcacagactcacacttcagtcatctggcttgggtcaacacggatcgtaagatgggcgggctgggacaaatgaacatccctatccttgcctatactaatcacgtaatctcccgtgcttatggtgtgttaaaagaggatgacggcattgcctaccgtggattgttcattattgatccgaagggtattttagggcaaatcacaattaacgaccttcctgttggccgttccgtggacgaaacactgcgcctgattcaggcgttccagtttgtcgataagcatggcgaggtgtgtccagcaaattggcatcccggttcagaaacgatcaaacccggtgttaaggaatctaaagcctattttgagaaacacattgcaggcaaagaccagttcaaaaatgcactgtataacttgttatctaagacaggggaaagcgaagatgaaatgcagcatttcaaacccattgaggacctttttcagtgctgcggacctacaaatgagactatggtacgttatatcgaaaacggtctttgtgaggacgaactgcgtaataagcctaactgctttgcggtgattagcgatcactttgattcatcacaaaaggattaa

SEQ ID NO: 21 - protein, 518 aa, Artificial sequence
  Synthetic polypeptide
MEEKVVELTHNWSAEQWDWPLQHNDEVIKVTNTNDKFEVGLDASFFTPKEIEVKVAGDNLVIHCRHESRAEHYGEIKREISRTYKLPSDVDTKTLTSNLTKRGHLVIAAKKKAMNKLLIAFGLVILFVTLPCVSESDEEFDDSAADDTDDSEAGGGSEGGDEYVTKGEFVETDGKKKECSSHEACYDQREPQAWCRLSENQAWTDRGCFCEDKLHSCVIERTNNGKLEYSYCAPEAGWQCAMTLAGSKAFIGQPAPNFKTTAVVNGDFKEISLGQFKGKYVVLLFYPLDFTFVCPTEIIAFSDRIAEFKQLDVAVMACSTDSHFSHLAWVNTDRKMGGLGQMNIPILAYTNHVISRAYGVLKEDDGIAYRGLFIIDPKGILGQITINDLPVGRSVDETLRLIQAFQFVDKHGEVCPANWHPGSETIKPGVKESKAYFEKHIAGKDQFKNALYNLLSKTGESEDEMQHFKPIEDLFQCCGPTNETMVRYIENGLCEDELRNKPNCFAVISDHFDSSQKD

SEQ ID NO: 22 - protein, 15 aa, Artificial sequence
  Synthetic peptide
GGGGSGGGGSGGGGS

SEQ ID NO: 23 - DNA, 36 bp, Artificial sequence
  Synthetic oligonucleotide
ggcggcggtagcggcggtggctctggcggtggttcc

SEQ ID NO: 24 - DNA, 36 bp, Artificial sequence
  Synthetic oligonucleotide
ggtggcggttctggtggcggctccggtggtggcagc

SEQ ID NO: 25 - DNA, 36 bp, Artificial sequence
  Synthetic oligonucleotide
ggtggtggctccggtggcggtagcggcggcggttct

SEQ ID NO: 26 - protein, 20 aa, Artificial sequence
  Synthetic peptide
  MISC_FEATURE (5)..(7): Xaa may be present or absent and when present is Gly
  MISC_FEATURE (8)..(8): Xaa may be present or absent and when present is Ser
  MISC_FEATURE (9)..(11): Xaa may be present or absent and when present is Gly
  MISC_FEATURE (12)..(12): Xaa may be present or absent and when present is Ser
  MISC_FEATURE (13)..(15): Xaa may be present or absent and when present is Gly
  MISC_FEATURE (16)..(16): Xaa may be present or absent and when present is Ser
  MISC_FEATURE (17)..(19): Xaa may be present or absent and when present is Gly
  MISC_FEATURE (20)..(20): Xaa may be present or absent and when present is Ser
GGGSXXXXXXXXXXXXXXXX

SEQ ID NO: 27 - protein, 25 aa, Artificial sequence
  Synthetic peptide
  MISC_FEATURE (6)..(9): Xaa is present or absent and when present is Gly
  MISC_FEATURE (10)..(10): Xaa is present or absent and when present is Ser
  MISC_FEATURE (11)..(14): Xaa is present or absent and when present is Gly
  MISC_FEATURE (15)..(15): Xaa is present or absent and when present is Ser
  MISC_FEATURE (16)..(19): Xaa is present or absent and when present is Gly
  MISC_FEATURE (20)..(20): Xaa is present or absent and when present is Ser
  MISC_FEATURE (21)..(24): Xaa is present or absent and when present is Gly
  MISC_FEATURE (25)..(25): Xaa is present or absent and when present is Ser
GGGGSXXXXXXXXXXXXXXXXXXXX

SEQ ID NO: 28 - protein, 12 aa, Artificial sequence
  Synthetic peptide
GGGSGGGSGGGS

SEQ ID NO: 29 - protein, 8 aa, Artificial sequence
  Synthetic peptide
WSHPQFEK

SEQ ID NO: 30 - protein, 10 aa, Artificial sequence
  Synthetic peptide
EQKLISEEDL

SEQ ID NO: 31 - protein, 8 aa, Artificial sequence
  Synthetic peptide
DYKDDDDK

SEQ ID NO: 32 - protein, 6 aa, Artificial sequence
  Synthetic peptide
HHHHHH

SEQ ID NO: 33 - protein, 9 aa, Artificial sequence
  Synthetic peptide
YPYDVPDYA

SEQ ID NO: 34 - protein, 6 aa, Artificial sequence
  Synthetic peptide
CCPGCC

(19) United States
(12) Patent Application Publication
(10) Pub. No.: US 2024/0115674 A1
(43) Pub. Date: Apr. 11, 2024

(54) VACCINE AND METHODS FOR PREVENTING FILARIASIS AND DIROFILARIASIS

(71) Applicants: THE BOARD OF TRUSTEES OF THE UNIVERSITY OF ILLINOIS, Urbana, IL (US); PAI LIFE SCIENCES INC., Seattle, WA (US)

(72) Inventors: Ramaswamy KALYANASUNDARAM, Rockford, IL (US); Darrick CARTER, Seattle, WA (US); Sean GRAY, Kent, WA (US)

(21) Appl. No.: 18/275,463

(22) PCT Filed: Feb. 2, 2022
(86) PCT No.: PCT/US2022/014894

§ 371 Date: Aug. 2, 2023

Related U.S. Application Data
(60) Provisional application No. 63/145,153, filed on Feb. 3, 2021.

(51) Int. Cl. A61K 39/00; A61K 9/00; A61P 33/00; C07K 14/435

(52) U.S. Cl. CPC A61K 39/0003; A61K 9/0019; A61P 33/00; C07K 14/4354; A61K 2039/54; A61K 2039/70; C12Y 111/01

(57) ABSTRACT

The present invention is a multivalent immunogenic composition for immunizing an animal against filariasis. In some aspects, the antigens of the multivalent immunogenic composition are protein-based, DNA-based, or a combination thereof. This invention also provides a method and kit for detecting a filarial nematode and determining vaccine efficacy.

### INTRODUCTION

This application claims the benefit of priority from U.S. Provisional Application Ser. No. 63/145,153, filed Feb. 3, 2021, the contents of which is incorporated herein by reference in its entireties.

This invention was made with government support under contract numbers AI116441, AI140708 and AI140708S awarded by the National Institutes of Health. The government has certain rights in the invention.

### BACKGROUND

Lymphatic filariasis caused by the filarial nematodes Wuchereria bancrofti, Brugia malayi, and Brugia timori, affects more than 120 million people worldwide (WHO (1992) World Health Organ. Tech. Rep. Ser. 821:1-71). A mass drug administration program by the World Health Organization, is significantly reducing the incidence rate of lymphatic filariasis in many parts of the world (Hotez (2009) Clin. Pharmacol. Ther. 85(6):659-64). Nevertheless, lack of effectiveness to the mass drug administration has been reported from several endemic regions mainly due to noncompliance (Babu & (2008) Trans. R. Soc. Trop. Med. Hyg. 102(12):1207-13; El-Setouhy, et al. (2007) Am. J. Trop. Med. Hyg. 77(6):1069-73). In addition, drug resistance has been reported to at least one of the drugs in the mass drug combination (Horton (2009) Ann. Trop. Med. Parasitol. 103(1):S33-40; Schwab, et al. (2007) Parasitology 134 (Pt 7):1025-40). Since yearly administration of the mass drugs is required for effective control, there is an alarming concern for selecting drug resistant parasites. Therefore, there is an immediate need for a multipronged approach in controlling this mosquito borne infection.

As with lymphatic filariasis, treatment of dirofilariasis (heartworm disease) in canids and felids has-included the use of macrolide agents such as ivermectin, milbemycin oxime, moxidectin and selamectin, which prevent larval development during the first 2 months after infection. However, these agents must be administered monthly for effectiveness and can be very expensive to a pet owner.

Vaccination is one strategy for controlling these infections and several subunit candidate vaccine antigens have been tested in laboratory animals with variable results (Bottazzi, et al. (2006) Expert Rev. Vaccines 5(2):189-98; Chenthamarakshan, et al. (1995) Parasite Immunol. 17(6):277-85; Dissanayake, et al. (1995) Am. J. Trop. Med. Hyg. 53(3):289-94; Li, et al. (1993) J. Immunol. 150(5):1881-5; Maizels, et al. (2001) Int. J. Parasitol. 31(9):889-98; Thirugnanam, et al. (2007) Exp. Parasitol. 116(4):483-91; Veerapathran, et al. (2009) PLoS Negl. Trop. Dis. 3(6):e457). Lymphatic filariasis is a multicellular organism with complex life cycle and produce large array of host modulatory molecules. Thus, fighting against this infection with a single antigen vaccine can be difficult. By screening a phage display cDNA expression library of the B. malayi parasite with sera from immune individuals, several potential vaccine candidates were identified (Gnanasekar, et al. (2004) Infect. Immun. 72(8):4707-15). However, a varying degree of protection was achieved with each of the candidate vaccine antigens when given as a DNA, protein or prime boost vaccine (Veerapathran, et al. (2009) supra).

Multivalent immunogenic compositions for immunizing an animal against filariasis and dirofilariasis are disclosed in U.S. Pat. No. 10,072,054, US 2019/0040108 and US 2020/0172585.

### SUMMARY OF THE INVENTION

This invention provides a multivalent immunogenic composition comprising a fusion of four or more antigens from one or more filarial nematodes wherein the fusion further comprises (i) a His tag; (ii) a linker between two or more of said antigens, e.g., GGGSGGGSGGGS (SEQ ID NO:28); (iii) replacement of one or more cysteine residues in said antigens with serine, or (iv) any combination of (i), (ii), and (iii), with the proviso that that when the fusion comprises (i) it further comprises one or both of (ii) or (iii). In some aspects, the filarial nematodes are selected from the group consisting of Brugia malayi, Wuchereria bancroft, Onchocerca volvulus, Loa, Brugia timori, and Dirofilaria immitis. In other aspects, the antigens are protein-based, DNA-based, or a combination thereof. In certain aspects, the antigens include Abundant Larval Transcript, Tetraspanin, Small Heat Shock Protein (HSP) 12.6, and Thioredoxin Peroxidase 2, or fragments thereof. In other aspects, all cysteine residues in the antigens are replaced with serine. In particular aspects, the fusion includes a GGGSGGGSGGGS (SEQ ID NO:28) linker between each of the antigens and all cysteine residues in the antigens are replaced with serine. Exemplary antigens of the fusion protein include antigens selected from the group of SEQ ID NO:1, SEQ ID NO:2, SEQ ID NO:3, SEQ ID NO:4, SEQ ID NO:5, SEQ ID NO:6, SEQ ID NO:7, and SEQ ID NO:8. Exemplary fusion proteins are selected from the group of SEQ ID NO:9, SEQ ID NO:10, SEQ ID NO:11, SEQ ID NO:12, SEQ ID NO:13, SEQ ID NO:14, and SEQ ID NO:15. In particular aspects, the multivalent immunogenic composition further includes an adjuvant. A recombinant vector including nucleic acids encoding a fusion protein of this invention as well as a recombinant host cell harboring said vector as also within the scope of this invention.

Methods for inducing an immune response and immunizing an animal against filariasis or dirofilariasis in a subject are also provided. These methods involve the step of administering the multivalent immunogenic composition of the invention to a subject thereby inducing an immune response in the subject and immunizing the subject against filariasis or dirofilariasis. In some aspects, the one or more additional doses of the immunogenic composition are administered to the subject. In other aspects, the multivalent immunogenic composition is administered by subcutaneous or intramuscular injection. In particular aspects, the multivalent immunogenic composition is administered with an adjuvant.

### BRIEF DESCRIPTION OF THE DRAWINGS

FIG. 1 shows the titer of BmHAXT-specific IgG antibodies in the sera of mice immunized three times with BmHAXT (tag-free), BmHAXT (ΔCys), BmHAXT (GS), and BmHAXT (ΔCys+GS). The titers were determined using an indirect ELISA. Sera from mouse immunized with adjuvant alone was used as the control.

FIG. 2 shows the levels of BmHAXT-specific antibody isotypes in the sera of mice immunized with BmHAXT (tag-free), BmHAXT (ΔCys), BmHAXT (GS), and BmHAXT (ΔCys+GS) proteins. *p<0.0001 compared to adjuvant control group analyzed by Kruskal-Wallis test.

FIG. 3 shows that compared to the adjuvant control groups, there was significant death of larva in the vaccinated groups. The percentage of protection was expressed as the number of dead parasites÷the number of total parasites recovered×100.

### DETAILED DESCRIPTION OF THE INVENTION

This invention is a multivalent immunogenic composition composed of a fusion protein of four or more antigens from one or more filarial nematodes wherein the fusion protein further includes (i) a His tag; (ii) a linker between two or more of said antigens; and/or (iii) replacement of one or more cysteine residues in said antigens with serine, provided that when the fusion protein includes the His tag it further includes one or both of a linker between two or more of the antigens or replacement of one or more cysteine residues in the antigens with serine. In certain aspects, antigens of the fusion protein are selected from the group of Abundant Larval Transcript (ALT2), Tetraspanin (TSP), Small Heat Shock Protein (HSP) and Thioredoxin Peroxidase 2 (TPX-2), or fragments thereof.

For the purposes of the present invention, a multivalent or polyvalent immunogenic composition refers to an immunogenic composition or vaccine prepared from several antigens. According to some aspects, the antigen is a nucleic acid molecule, which is referred to herein as a “DNA-based” antigen. According to other aspects, the antigen is a protein or polypeptide, which is referred to herein as “protein-based” antigen. A multivalent immunogenic composition of the invention can be composed of two, three, four, five, six or up to ten antigens or their fragments in various permutation combinations. In particular aspects, the multivalent immunogenic composition is composed of two, three or four antigens. In some aspects, the multivalent immunogenic composition is composed of solely of protein antigens. In other aspects, the multivalent immunogenic composition is composed solely of DNA-based antigens. In yet other aspects, the multivalent immunogenic composition is composed of a mixture of protein- and DNA-based antigens.

Antigens of the multivalent immunogenic composition of this invention are covalently attached to form a hybrid or chimeric molecule or fusion protein. In some aspects, the antigens may be immediately adjacent to one another. In other aspects, two or more of the antigens are linked to one another via a peptide linker. In particular aspects, all four antigens are linked via peptide linkers. Peptide linkers of this invention are ideally composed of one to about 20 amino acid residues. The term “peptide linking group,” “peptide linker,” or “linker” is meant to refer to a peptide moiety that acts as a molecular bridge to operably link two different antigens together. Desirably, the linkers of this invention are composed of glycine or serine, or a combination thereof. It is desirable that this linker is a flexible linker. The flexible linker preferably has a length of one to about 20 amino acid residues, particularly a length of 4, 5, 8, 10, 12, 15, 16 or 20 amino residues. The flexible linker is preferably a glycine/serine linker, i.e., a peptide linker composed primarily of the amino acids glycine and serine. In one aspect, the linker is a (GGGS)ₙ linker (SEQ ID NO:26) or (GGGGS)ₙ (SEQ ID NO:27), wherein n is 1 to 5. In some aspects, the linker has the amino acid sequence GGGGSGGGGSGGGGS (SEQ ID NO:22). In other aspects, the linker has the amino acid sequence GGGSGGGSGGGS (SEQ ID NO:28). In some aspects, a linker is disposed between each of the different antigens.

In one aspect, the antigens of the multivalent immunogenic composition are different proteins from one species of filarial nematode. As an example of this aspect, the multivalent immunogenic composition is composed of ALT2, HSP, TSP and TPX2 antigens isolated from one or more strains of B. malayi or D. immitis. In yet a further aspect, the multivalent immunogenic composition is composed of a combination of different antigens from different species of filarial nematodes. By way of illustration, the multivalent immunogenic composition can be composed of the ALT2 antigen from B. malayi, HSP from B. malayi, TSP from L. loa and TPX2 from D. immitis.

For preparing multivalent DNA-based or multivalent recombinant DNA-based immunogenic composition, the DNA sequence of the gene of interest (also used interchangeably as DNA molecule) need not contain the full length of DNA encoding the corresponding protein. Likewise, when preparing fusion protein-based or multivalent recombinant protein immunogenic compositions, the protein sequence need not contain the full-length protein. In most cases, a fragment of the protein or gene which encodes an epitope region is sufficient for immunization. The DNA/protein sequence of an epitope region can be found by sequencing the corresponding part of the gene from various strains or species and comparing them. The major antigenic determinants are likely to be those showing the greatest heterology. Also, these regions are likely to lie accessibly in the conformational structure of the proteins. One or more such fragments of proteins or genes encoding the antigenic determinants can be prepared by chemical synthesis or by recombinant DNA technology.

As described herein, the ALT2, TSP, TPX2, and HSP antigens were identified as providing protection against infection by filaria larvae. Accordingly, in particular aspects, the instant immunogenic composition includes the ALT2, TSP, TPX2, and HSP protein antigens and/or nucleic acid molecules encoding the ALT2, TSP, TPX2, and HSP protein, or fragments thereof. Protein and nucleic acid sequences for these antigens are available under the GENBANK accession numbers and/or sequences listed in Table 1.

TABLE 1

| Antigen | Source | Protein | Nucleic Acid |
|---|---|---|---|
| ALT2 | B. malayi | P90708 | BMU84723 |
| | W. bancrofti | AAC35355 | AF084553 |
| | L. loa | XP_003151340 | XM_003151292 |
| | D. immitis | AAC47031 | — |
| TSP | B. malayi | ABN55911 | EF397425 |
| | L. loa | XP_003136177 | XM_003136129 |
| HSP | B. malayi | AAU04396 | AY692227 |
| | O. volvulus | CAA48633 | X68669 |
| | L. loa | XP_003139338 | XM_003139290 |
| | D. immitis | QHA79233 | — |
| TPX2 | B. malayi | Q17172 | U47100 |
| | D. immitis | AAC38831 | — |

In addition, the nucleotide sequence encoding O. volvulus TSP can be found under GENBANK Accession No. JN861043. The protein antigens and nucleic acid molecules of the invention can be used as full-length molecules. Exemplary wild-type protein sequences for HSP, ALT2, and TPX2 protein sequences are respectively set forth in SEQ ID NOs:1, 2, and 3. Alternatively, the antigens may be truncated at the N- and/or C-terminus. In this respect, the present invention further includes the use of fragments of the above-referenced protein antigens and nucleic acid molecules. Fragments are defined herein as 20, 30, 40, 50, 60, 70, 80, 90, 100, 150, or 200 amino acid residue portions of full-length protein antigens (e.g., those listed in Table 1) or 60, 90, 120, 150, 180, 210, 240, 270, 300, 350, or 600 nucleotide portion of full-length nucleic acid molecules (e.g., those listed in Table 1). An exemplary protein fragment includes the Large Extracellular Loop of TSP, which is set forth herein under SEQ ID NO:4.

With respect to certain aspects of the invention, the multivalent immunogenic composition of the invention includes other known antigens from filarial nematodes. Examples of other suitable antigens include, but are not limited to, glutathione peroxidase (see Cookson, et al. (1992) Proc. Natl. Acad. Sci. USA 89:5837-5841; Maizels, et al. (1983) Parasitology 87:249-263; Maizels, et al. (1983) Clin. Exp. Immunol. 51:269-277); recombinant antigen (BmRi; see Noordin, et al. (2004) Filaria J. 3:10); class II aminoacyl-tRNA synthetase (see Kron, et al. (1995) FEBS Lett. 374:122-4); heat shock cognate 70 (hsc70) protein (see Selkirk, et al. (1989) J. Immunol. 143:299-308); paramyosin (see Li, et al. (1991) Mol. Biochem. Parasitol. 49:315-23); tropomyosin (Hartmann, et al. (2006) Vaccine 24(17):3581-90); chitinase (Adam, et al. (1996) J. Biol. Chem. 271(3):1441-7); Abundant Larval Transcript (ALT)-1 (Gregory, et al. (2000) Infect. Immun. 68(7):4174-9); immunodominant hypodermal antigen SPX1 (Bradley, et al. (1993) Exp. Parasitol. 77(4):414-424). In some aspects, the antigen is obtained from a filarial nematode selected from the group of W. bancrofti, B. malayi, O. volvulus, L. loa, D. immitis and B. timori. In certain aspects, the antigen is B. malayi or Dirofilaria tropomyosin, or a fragment thereof; B. malayi or Dirofilaria chitinase, or a fragment thereof; B. malayi or Dirofilaria ALT-1, or a fragment thereof; B. malayi or Dirofilaria SPX1, or a fragment thereof; B. malayi or D. immitis venom allergen antigen 5-like protein, or a fragment thereof; B. malayi or D. immitis Macrophage migration Inhibitory Factor (MIF)-1 protein, or a fragment thereof; B. malayi or Dirofilaria MIF-2 protein, or a fragment thereof; or B. malayi or Dirofilaria cystatin protein, or a fragment thereof.

According to the present invention, the antigens of the fusion protein and immunogenic composition are isolated from a filarial nematode. In this respect, an isolated nucleic acid molecule or protein is a nucleic acid molecule or protein that has been removed from its natural milieu (i.e., that has been subjected to human manipulation). As such, “isolated” does not reflect the extent to which the nucleic acid molecule or protein has been purified. In particular aspects, the antigens are purified (e.g., purified to greater than 95% homogeneity). An isolated and optionally purified nucleic acid molecule or protein of the present invention can be obtained from its natural source or produced using recombinant DNA technology (e.g., polymerase chain reaction (PCR) amplification or cloning) or chemical synthesis. Isolated nucleic acid molecules and proteins can also include, for example, natural allelic variants or isomers that induce an immune response in the host.

One aspect of the present invention includes a recombinant vector, which includes at least one isolated nucleic acid molecule of the present invention, inserted into a vector capable of delivering the nucleic acid molecule into a host cell. Such a vector contains heterologous nucleic acid sequences, that are nucleic acid sequences that are not naturally found adjacent to nucleic acid molecules of the present invention and that preferably are derived from a species other than the species from which the nucleic acid molecule(s) are derived. The vector can be either prokaryotic or eukaryotic, and typically is a virus or a plasmid. Recombinant vectors can be used in the cloning, sequencing, and/or otherwise manipulating the nucleic acid molecules of the present invention.

The present invention also includes an expression vector, which includes a nucleic acid molecule of the present invention in a recombinant vector that is capable of expressing the nucleic acid molecule when transformed into a host cell. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, parasite, insect, other animal, and plant cells. Preferred expression vectors of the present invention can direct gene expression in bacterial, yeast, helminth or other parasite, insect and mammalian cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, helminth or other endoparasite, or insect and mammalian cells, such as, but not limited to, tac, lac, trp, trc, oxy-pro, omp/lpp, rrnB, bacteriophage lambda (such as lambda p_{L} and lambda p_{R} and fusions that include such promoters), bacteriophage T7, T7lac, bacteriophage T3, bacteriophage SP6, bacteriophage SP01, metallothionein, alpha-mating factor, Pichia alcohol oxidase, alphavirus subgenomic promoter, antibiotic resistance gene, baculovirus, Heliothis zea insect virus, vaccinia virus, herpesvirus, raccoon poxvirus, other poxvirus, adenovirus, cytomegalovirus (such as immediate early promoter), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with parasitic helminths, such as W. bancrofti, B. malayi or D. immitis transcription control sequences.

Recombinant molecules of the present invention may also contain (a) secretory signals (i.e., signal segment nucleic acid sequences) to enable an expressed protein of the present invention to be secreted from the cell that produces the protein and/or (b) fusion sequences which lead to the expression of nucleic acid molecules of the present invention as fusion proteins. Examples of suitable signal segments include any signal segment capable of directing the secretion of a protein of the present invention. Preferred signal segments include, but are not limited to, tissue plasminogen activator (t-PA), interferon, interleukin, growth hormone, histocompatibility and viral envelope glycoprotein signal segments. In addition, a nucleic acid molecule of the present invention can be joined to a fusion segment that directs the encoded protein to the proteosome, such as a ubiquitin fusion segment. Eukaryotic recombinant molecules may also include intervening and/or untranslated sequences surrounding and/or within the nucleic acid sequences of nucleic acid molecules of the present invention.

Another aspect of the present invention includes a recombinant host cell harboring one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a nucleic acid molecule of the present invention. Host cells can be either untransformed cells or cells that are already transformed with at least one nucleic acid molecule (e.g., nucleic acid molecules encoding one or more proteins of the present invention and/or other proteins useful in the production of multivalent immunogenic compositions). Host cells of the present invention either can be endogenously (i.e., naturally) capable of producing proteins of the present invention or can be capable of producing such proteins after being transformed with at least one nucleic acid molecule of the present invention. Host cells of the present invention can be any cell capable of producing at least one protein of the present invention, and include bacterial, fungal (including yeast), parasite (including helminth, protozoa and ectoparasite), other insect, other animal and plant cells. Preferred host cells include bacterial, mycobacterial, yeast, helminth, insect and mammalian cells. More preferred host cells include Salmonella, Escherichia, Bacillus, Listeria, Saccharomyces, Spodoptera, Mycobacteria, Trichoplusia, BHK (baby hamster kidney) cells, MDCK cells (Madin-Darby canine kidney cell line), CRFK cells (Crandell feline kidney cell line), CV-1 cells (African monkey kidney cell line used, for example, to culture raccoon poxvirus), COS (e.g., COS-7) cells, and Vero cells. Particularly preferred host cells are Escherichia coli, including E. coli K-12 derivatives; Salmonella typhi; Salmonella typhimurium; Spodoptera frugiperda; Trichoplusia ni; BHK cells; MDCK cells; CRFK cells; CV-1 cells; COS cells; Vero cells; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells. In one aspect, the proteins may be expressed as heterologous proteins in myeloma cell lines employing immunoglobulin promoters.

A recombinant cell is preferably produced by transforming a host cell with one or more recombinant molecules, each comprising a nucleic acid molecule of the present invention and one or more transcription control sequences, examples of which are disclosed herein.

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein. Moreover, while non-codon-optimized sequences may be used to express fusion proteins in host cells such as E. coli, in aspects pertaining to DNA vaccines, the nucleic acid molecule may be codon-optimized to facilitate expression in mammalian cells. Moreover, to facilitate expression of one or more of the recombinant proteins in a recombinant host cell, the protein sequence can be manipulated. By way of illustration, the insertion of a glycine residue after the N-terminal methionine residue of the B. malayi ALT2 protein was found to improve expression of this protein in E. coli.

Isolated protein-based antigens of the present invention can be produced in a variety of ways, including production and recovery of natural proteins, production and recovery of recombinant proteins, and chemical synthesis of the proteins. In one aspect, an isolated protein of the present invention is produced by culturing a cell capable of expressing the protein under conditions effective to produce the protein, and recovering the protein. A preferred cell to culture is a recombinant cell of the present invention. Effective culture conditions include, but are not limited to, effective media, bioreactor, temperature, pH and oxygen conditions that permit protein production. An effective, medium refers to any medium in which a cell is cultured to produce a protein of the present invention. Such medium typically includes an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

Recovery of proteins of invention can include collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins of the present invention are preferably retrieved in substantially pure form thereby allowing for the effective use of the protein as a therapeutic composition. A therapeutic composition for animals, for example, should exhibit no substantial toxicity and preferably should be capable of stimulating the production of antibodies in a treated animal.

In some aspects, the fusion protein includes a “purification tag,” “affinity tag,” or “tag” at its N-terminus or C-terminus. Suitable tags include the peptides: WSHPQFEK (SEQ ID NO:29) available under the tradename STREP-TAG® II, EQKLISEEDL (SEQ ID NO:30) known as a myc-tag, DYKDDDDK (SEQ ID NO:31) available under the tradename FLAG®-tag, HHHHHH (SEQ ID NO:32) known as a His-tag, YPYDVPDYA (SEQ ID NO:33) known as an HA-tag, CCPGCC (SEQ ID NO:34) known as a TC-tag, or AAA known as a 3×Ala-tag; or proteins such as glutathione-S-transferase (GST), maltose binding protein (MBP), or chitin binding domain (CBD), which also allow for easy detection and/or easy purification of recombinant proteins. Further, proteins with chromogenic or fluorescent properties, such as green fluorescent protein (GFP) or yellow fluorescent protein (YFP), are also suitable tags of the present disclosure. In a particular aspect, the fusion protein includes an N-terminal His-tag.

To further facilitate recombinant expression and purification of the fusion protein of this invention, certain aspects provide that the one or more cysteine residues in the antigens of the fusion protein are replaced with serine residues. In particular aspects all of the cysteine residues in the fusion protein are replaced with serine residues. Exemplary protein sequences for HSP, ALT2, TPX2 and TSP protein sequences, wherein all cysteine residues have been mutated to serine residues, are respectively set forth in SEQ ID NOs:5, 6, 7 and 8.

Recombinant fusion proteins of this invention include, but are not limited to, fusion proteins composed of four or more antigens, wherein the fusion protein further includes a His tag, a linker between two or more of said antigens, and/or one or more cysteine residues in said antigens are replaced with serine residues. Exemplary fusion proteins of this invention include BmHAXT (his-tagged) (SEQ ID NO:9), which includes an N-terminal His-tag; BmHAXT (ΔCys) (SEQ ID NO:10), wherein all cysteines (17 total) have been mutated to serine residues; BmHAXT (GS) (SEQ ID NO:11), wherein three separate 15 amino acid glycine/serine linkers have been inserted between each of the four antigens; BmHAXT (ΔCys+GS) (SEQ ID NO:12), wherein all cysteines (17 total) have been mutated to serine residues and three separate 15 amino acid glycine/serine linkers have been inserted between each of the four antigens); BmHAXT (his-tagged+ΔCys) (SEQ ID NO:13), which includes an N-terminal His-tag and replacement of all cysteines (17 total) with serine residues; BmHAXT (his-tagged+GS) (SEQ ID NO:14), which includes an N-terminal His-tag and three separate 15 amino acid glycine/serine linkers have been inserted between each of the four antigens; BmHAXT (his-tagged+ΔCys+GS) (SEQ ID NO:15), which includes an N-terminal His-tag, replacement of all cysteines (17 total) with serine residues, and three separate 15 amino acid glycine/serine linkers have been inserted between each of the four antigens. Nucleic acids encoding exemplary proteins are as follows: BmHAXT (his-tagged) (SEQ ID NO:16), BmHAXT (ΔCys) (SEQ ID NO:17), BmHAXT (GS) (SEQ ID NO:18), and BmHAXT (ΔCys+GS) (SEQ ID NO:19).

One aspect of the present invention is an immunogenic composition or vaccine that, when administered to an animal in an effective manner, is capable of inducing an immune response and ideally protecting that animal from filariasis or dirofilariasis caused by a nematode such as a B. malayi or D. immitis. In some aspects, the invention provides a method for treating or protecting an animal from a disease caused by a filarial nematode. In other aspects, the invention provides a method for treating or protecting an animal, e.g., a dog or cat, from dirofilariasis (heartworm disease). Immunogenic compositions include antigenic molecules such as an isolated antigenic protein of the present invention, an isolated nucleic acid molecule of the present invention, and hybrids and mixtures thereof. As used herein, the multivalent immunogenic composition of the invention induces an immune response when administered in an effective manner to an animal such as a human, cat or dog thereby treating, ameliorating, and/or preventing disease caused by a filarial or dirofilarial nematode including, but not limited to, W. bancrofti, B. malayi, O. volvulus, L. loa, D. immitis, D. repens, Mansonella streptocerca, Dracunculus medinensis, M. perstans, M. ozzardi, and/or B. timori. Immunogenic composition of the present invention can be administered to any animal susceptible to such therapy, preferably to mammals, and more preferably to humans, pets such as dogs and cats, and economic food animals and/or zoo animals.

To induce an immune response and protect an animal against infection by a filarial or dirofilarial nematode, an immunogenic composition of the present invention is administered to the animal in an effective manner such that the composition elicits a cell-mediated immune response and/or the production of antibodies that specifically bind to the antigens of the immunogenic composition and protect that animal from a disease caused by the filarial or dirofilarial nematode. Compositions of the present invention can be administered to animals prior to infection in order to prevent infection (i.e., as a preventative vaccine) and/or can be administered to animals after infection in order to treat disease caused by the filarial or dirofilarial nematode (i.e., as a therapeutic vaccine).

Compositions of the present invention can be formulated in an excipient that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one aspect of the present invention, the immunogenic composition includes an adjuvant. An “adjuvant,” as defined herein, is a substance that serves to enhance the immunogenicity of an immunogenic composition of the invention. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan.

Suitable adjuvants to enhance effectiveness of the immunogenic composition include, but are not limited to:
(1) aluminum salts (alum), such as aluminum hydroxide, aluminum phosphate, aluminum sulfate, etc.;
(2) calcium-based salts;
(3) silica;
(4) oil-in-water emulsion formulations (with or without other specific immunostimulating agents such as muramyl peptides (defined below) or bacterial cell wall components), such as, for example,
   (a) MF59 (WO 90/14837), containing 5% squalene, 0.5% polysorbate 80, and 0.5% sorbitan trioleate (optionally containing various amounts of muramyl tripeptide phosphatidylethanolamine) formulated into submicron particles using a microfluidizer such as Model 110Y microfluidizer (Microfluidics, Newton, MA),
   (b) SAF, containing 10% squalene, 0.4% polysorbate 80, 5% pluronic-blocked polymer L121, and thr-MDP either microfluidized into a submicron emulsion or vortexed to generate a larger particle size emulsion,
   (c) Ribi™, adjuvant system (RAS), (Corixa, Hamilton, MT) containing 2% squalene, 0.2% polysorbate 80, and one or more bacterial cell wall components from the group consisting of 3-O-deacylated monophosphorylipid A (MPL™) described in U.S. Pat. No. 4,912,094, trehalose dimycolate (TDM), and cell wall skeleton (CWS), preferably MPL+CWS (Detox™); and
   (d) a Montanide ISA;
(5) saponin adjuvants, such as those sold under the tradenames QUIL-A® or QS-21 STIMULON® (Antigenics, Framingham, MA) (see, e.g., U.S. Pat. No. 5,057,540), may be used or particles generated therefrom such as ISCOM (immunostimulating complexes formed by the combination of cholesterol, saponin, phospholipid, and amphipathic proteins) and Iscomatrix™ (having essentially the same structure as an ISCOM but without the protein);
(6) bacterial components (e.g., endotoxins, in particular superantigens, exotoxins and cell wall components) and lipopolysaccharides, synthetic lipid A analogs such as aminoalkyl glucosamine phosphate compounds (AGP), or derivatives or analogs thereof, which are available from Corixa, and described in U.S. Pat. No. 6,113,918; one such AGP is 2-[(R)-3-tetradecanoyloxytetradecanoylamino]ethyl 2-Deoxy-4-O-phosphono-3-O—[(R)-3-tetradecanoyloxy-tetradecanoyl]-2-[(R)-3-tetradecanoyloxytetradecanoylamino]-b-D-glucopyranoside, which is also known as 529 (formerly known as RC529), which is formulated as an aqueous form or as a stable emulsion;
(7) synthetic polynucleotides such as oligonucleotides containing CpG motif(s) (U.S. Pat. No. 6,207,646);
(8) cytokines and chemokines (e.g., granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), IL-3, IL-4, IL-5, IL-6, IL-7, IL-8, IL-10, IL-12, IL-15, IL-18, interferon gamma, interferon gamma inducing factor I (IGIF), transforming growth factor beta, RANTES (regulated upon activation, normal T-cell expressed and presumably secreted), macrophage inflammatory proteins (e.g., MIP-1 alpha and MIP-1 beta), tumor necrosis factor (TNF), costimulatory molecules B7-1 and B7-2, and Leishmania elongation initiating factor (LEIF));
(9) complement, such as a trimer of complement component C3d;
(10) toll-like receptor agonists, e.g., TLR4 agonists such as glucopyranosyl lipid adjuvant (GLA);
(11) serum proteins, e.g., transferrin;
(12) viral coat proteins, e.g., rotavirus capsid VP6 protein; and
(13) block copolymer adjuvants, e.g., Hunter's TITERMAX® adjuvant (VAXCEL, Inc. Norcross, GA).

Muramyl peptides include, but are not limited to, N-acetyl-muramyl-L-threonyl-D-isoglutamine (thr-MDP), N-acetyl-normuramyl-L-alanine-2-(1′-2′dipalmitoyl-sn-glycero-3-hydroxyphosphoryloxy)-ethylamine (MTP-PE), etc.

Protein adjuvants of the present invention can be delivered in the form of the protein themselves or of nucleic acid molecules encoding such proteins using the techniques described herein.

In certain aspects, the adjuvant includes an aluminum salt. The aluminum salt adjuvant may be an alum-precipitated vaccine or an alum-adsorbed vaccine. Aluminum-salt adjuvants are well-known in the art and are described, for example, in Harlow & Lane ((1988) Antibodies: A Laboratory Manual, Cold Spring Harbor Laboratory) and Nicklas ((1992) Res. Immunol. 143:489-493). The aluminum salt includes, but is not limited to, hydrated alumina, alumina hydrate, alumina trihydrate (ATH), aluminum hydrate, aluminum trihydrate, aluminum (III) hydroxide, aluminum hydroxyphosphate sulfate, Aluminum Phosphate Adjuvant (APA), amorphous alumina, trihydrated alumina, or trihydroxyaluminum.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and steam sterilized.

In certain aspects, a commercially available Al(OH)₃ (e.g., aluminum hydroxide gel sold under the tradename ALHYDROGEL®) is used to adsorb proteins in a ratio of 50-200 μg protein/mg aluminum hydroxide. Adsorption of protein is dependent, in another aspect, on the pI (Isoelectric pH) of the protein and the pH of the medium. A protein with a lower pI adsorbs to the positively charged aluminum ion more strongly than a protein with a higher pI. Aluminum salts may establish a depot of antigen that is released slowly over a period of 2-3 weeks, be involved in nonspecific activation of macrophages and complement activation, and/or stimulate innate immune mechanism (possibly through stimulation of uric acid). See, e.g., Lambrecht, et al. (2009) Curr. Opin. Immunol. 21:23.

In some aspects, the adjuvant is a mixture of 2, 3, or more of the above adjuvants, e.g., SBAS2 (an oil-in-water emulsion also containing 3-deacylated monophosphoryl lipid A and QS-21); or alum in combination with GLA (AL019).

The multivalent immunogenic composition of the invention can be formulated as single dose vials, multi-dose vials or as pre-filled glass or plastic syringes.

In one aspect, multivalent immunogenic compositions of the present invention are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

Pharmaceutically acceptable carriers for liquid formulations are aqueous or non-aqueous solutions, suspensions, emulsions or oils. Examples of nonaqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

The pharmaceutical composition may be isotonic, hypotonic or hypertonic. However, it is often preferred that a composition for infusion or injection is essentially isotonic, when it is administrated. Hence, storage of the composition may preferably be isotonic or hypertonic. If the composition is hypertonic for storage, it may be diluted to become an isotonic solution prior to administration.

The isotonic agent may be an ionic isotonic agent such as a salt or a non-ionic isotonic agent such as a carbohydrate. Examples of ionic isotonic agents include but are not limited to NaCl, CaCl₂), KCl and MgCl₂. Examples of non-ionic isotonic agents include but are not limited to mannitol, sorbitol and glycerol.

It is also preferred that at least one pharmaceutically acceptable additive is a buffer. For some purposes, for example, when the composition is meant for infusion or injection, it is often desirable that the composition includes a buffer, which is capable of buffering a solution to a pH in the range of 4 to 10, such as 5 to 9, for example 6 to 8.

The buffer may, for example, be selected from Tris, acetate, glutamate, lactate, maleate, tartrate, phosphate, citrate, carbonate, glycinate, histidine, glycine, succinate and triethanolamine buffer. The buffer may be selected from USP compatible buffers for parenteral use, in particular, when the formulation is for parenteral use. For example, the buffer may be selected from the group of monobasic acids such as acetic, benzoic, gluconic, glyceric and lactic; dibasic acids such as aconitic, adipic, ascorbic, carbonic, glutamic, malic, succinic and tartaric, polybasic acids such as citric and phosphoric; and bases such as ammonia, diethanolamine, glycine, triethanolamine, and Tris.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, Polysorbate 80 (PS-80), Polysorbate 20 (PS-20), and Poloxamer 188 (P188) are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

The formulations of the invention may also contain a surfactant. Preferred surfactants include, but are not limited to, the polyoxyethylene sorbitan esters surfactants, especially PS-20 and PS-80; copolymers of ethylene oxide (EO), propylene oxide (PO), and/or butylene oxide (BO), sold under the tradename DOWFAX™, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters, such as sorbitan trioleate and sorbitan monolaurate. A preferred surfactant for including in the emulsion is PS-80.

Mixtures of surfactants can be used. A combination of a polyoxyethylene sorbitan ester such as polyoxyethylene sorbitan monooleate (PS-80) and an octoxynol such as t-octylphenoxypolyethoxyethanol is also suitable. Another useful combination comprises laureth 9 plus a polyoxyethylene sorbitan ester and/or an octoxynol.

Poloxamer may also be used in the compositions of the invention. A poloxamer is a nonionic triblock copolymer composed of a central hydrophobic chain of polyoxypropylene (poly(propylene oxide)) flanked by two hydrophilic chains of polyoxyethylene (poly(ethylene oxide)). Poloxamers are also known by the tradename PLURONIC®. Because the lengths of the polymer blocks can be customized, many different poloxamers exist that have slightly different properties. For the generic term “poloxamer”, these copolymers are commonly named with the letter “P” (for poloxamer) followed by three digits, the first two digits×100 give the approximate molecular mass of the polyoxypropylene core, and the last digit ×10 gives the percentage polyoxyethylene content (e.g., P407=Poloxamer with a polyoxypropylene molecular mass of 4,000 g/mol and a 70% polyoxyethylene content). For the PLURONIC® tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit ×10 gives the percentage polyoxyethylene content (e.g., L61 is a PLURONIC® with a polyoxypropylene molecular mass of 1,800 g/mol and a 10% polyoxyethylene content). See U.S. Pat. No. 3,740,421.

Preferably, the poloxamer generally has a molecular weight in the range from 1100 to 17,400 Da, from 7,500 to 15,000 Da, or from 7,500 to 10,000 Da. The poloxamer can be selected from poloxamer 188 or poloxamer 407. The final concentration of the poloxamer in the formulations is from 0.001% to 5% weight/volume, or 0.025% to 1% weight/volume. In certain aspects, the polyol is propylene glycol and is at final concentration from 1% to 20% weight/volume. In certain aspects, the polyol is polyethylene glycol 400 and is at final concentration from 1% to 20% weight/volume.

Suitable polyols for the formulations of the invention are polymeric polyols, particularly polyether diols including, but are not limited to, propylene glycol and polyethylene glycol, Polyethylene glycol monomethyl ethers. Propylene glycol is available in a range of molecular weights of the monomer from about 425 to about 2700. Polyethylene glycol and Polyethylene glycol monomethyl ether is also available in a range of molecular weights ranging from about 200 to about 35000 including but not limited to PEG200, PEG300, PEG400, PEG1000, PEG MME 550, PEG MME 600, PEG MME 2000, PEG MME 3350 and PEG MME 4000. A preferred polyethylene glycol is polyethylene glycol 400. The final concentration of the polyol in the formulations of the invention may be 1% to 20% weight/volume or 6% to 20% weight/volume.

The formulation may also contain a pH-buffered saline solution. The buffer may, for example, be selected from the group consisting of Tris, acetate, glutamate, lactate, maleate, tartrate, phosphate, citrate, carbonate, glycinate, histidine, glycine, succinate, HEPES (4-(2-hydroxyethyl)-1-piperazineethanesulfonic acid), MOPS (3-(N-morpholino)propanesulfonic acid), MES (2-(N-morpholino)ethanesulfonic acid) and triethanolamine buffer. The buffer is capable of buffering a solution to a pH in the range of 4 to 10, 5.2 to 7.5, or 5.8 to 7.0. In certain aspects of the invention, the buffer is selected from the group of phosphate, succinate, histidine, MES, MOPS, HEPES, acetate or citrate. The buffer may furthermore, for example, be selected from USP compatible buffers for parenteral use, in particular, when the pharmaceutical formulation is for parenteral use. The concentrations of buffer will range from 1 mM to 100 mM. The concentrations of buffer will range from 10 mM to 80 mM. The concentrations of buffer will range from 1 mM to 50 mM or 5 mM to 50 mM.

While the saline solution (i.e., a solution containing NaCl) is preferred, other salts suitable for formulation include but are not limited to, CaCl₂, KCl and MgCl₂ and combinations thereof. Non-ionic isotonic agents including but not limited to sucrose, trehalose, mannitol, sorbitol, and glycerol may be used in lieu of a salt. Suitable salt ranges include, but are not limited to, 25 mM to 500 mM or 40 mM to 170 mM. In one aspect, the saline is NaCl, optionally present at a concentration from 20 mM to 170 mM.

In some aspects, the composition of the invention is administered to a subject by one or more methods known to a person skilled in the art, such as parenterally, transmucosally, transdermally, intramuscularly, intravenously, intra-dermally, intra-nasally, subcutaneously, intra-peritonealy, and formulated accordingly. In one aspect, a composition of the present invention is administered via epidermal injection, intramuscular injection, intravenous, intra-arterial, subcutaneous injection, or intra-respiratory mucosal injection of a liquid preparation. Liquid formulations for injection include solutions and the like.

One aspect of the present invention is a controlled release formulation that is capable of slowly releasing a composition of the present invention into an animal. As used herein, a controlled release formulation includes a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

A preferred controlled release formulation is capable of releasing an immunogenic composition of the present invention into the blood of the treated animal at a constant rate sufficient to attain therapeutic dose levels of the composition to protect an animal from disease caused by a filarial or dirofilarial nematode. For example, the immunogenic composition can be administered using intravenous infusion, a transdermal patch, liposomes, or other modes of administration. In another aspect, polymeric materials are used, e.g., in microspheres in or an implant. The immunogenic composition is preferably released over a period of time ranging from about 1 to about 12 months. A controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

Immunogenic compositions or vaccines of the present invention can be administered to animals prior to infection in order to prevent infection and/or can be administered to animals after infection in order to treat disease caused by a filarial nematode. For example, proteins, nucleic acids and mixtures thereof can be used as immunotherapeutic agents. Acceptable protocols to administer compositions in an effective manner include individual dose size, number of doses, frequency of dose administration, and mode of administration. Determination of such protocols can be accomplished by those skilled in the art. A suitable single dose is a dose that is capable of protecting an animal from disease when administered one or more times over a suitable time period. For example, a preferred single dose of a protein-based vaccine is from about 1 microgram (pg) to about 10 milligrams (mg) of protein-based vaccine per kilogram body weight of the animal. Booster vaccinations can be administered from about 2 weeks to several years after the original administration. Booster administrations preferably are administered when the immune response of the animal becomes insufficient to protect the animal from disease. A preferred administration schedule is one in which from about 10 μg to about 1 mg of the vaccine per kg body weight of the animal is administered from about one to about two times over a time period of from about 2 weeks to about 12 months. Modes of administration can include, but are not limited to, subcutaneous, intradermal, intravenous, intranasal, oral, transdermal and intramuscular routes.

Wherein the immunogenic composition includes a nucleic acid molecule, the immunogenic composition can be administered to an animal in a fashion to enable expression of that nucleic acid molecule into a protective protein in the animal. Nucleic acid molecules can be delivered to an animal in a variety of methods including, but not limited to, administering a naked (i.e., not packaged in a viral coat or cellular membrane) nucleic acid as a genetic vaccine (e.g., as naked DNA molecules, such as is taught, for example in Wolff, et al. (1990) Science 247:1465-1468); or administering a nucleic acid molecule packaged as a recombinant virus vaccine or as a recombinant cell vaccine (i.e., the nucleic acid molecule is delivered by a viral or cellular vehicle).

A genetic (i.e., naked nucleic acid) vaccine of the present invention includes a nucleic acid molecule of the present invention and preferably includes a recombinant molecule of the present invention that preferably is replication, or otherwise amplification, competent. A genetic vaccine of the present invention can include one or more nucleic acid molecules of the present invention in the form of, for example, a dicistronic recombinant molecule. Preferred genetic vaccines include at least a portion of a viral genome (i.e., a viral vector). Preferred viral vectors include those based on alphaviruses, poxviruses, adenoviruses, herpesviruses, picornaviruses, and retroviruses, with those based on alphaviruses (such as sindbis or Semliki forest virus), species-specific herpesviruses and poxviruses being particularly preferred. Any suitable transcription control sequence can be used, including those disclosed as suitable for protein production. Particularly preferred transcription control sequences include cytomegalovirus immediate early (preferably in conjunction with Intron-A), Rous sarcoma virus long terminal repeat, and tissue-specific transcription control sequences, as well as transcription control sequences endogenous to viral vectors if viral vectors are used. The incorporation of a “strong” polyadenylation signal is also preferred.

Genetic vaccines of the present invention can be administered in a variety of ways, including intramuscular, subcutaneous, intradermal, transdermal, intranasal and oral routes of administration. Moreover, it is contemplated that the vaccine can be delivered by gene gun, skin patch, electroporation, or nano-based delivery. In this respect, DNA-based and protein-based vaccines can be administered at the same time. A preferred single dose of a genetic vaccine ranges from about 1 nanogram (ng) to about 600 μg, depending on the route of administration and/or method of delivery, as can be determined by those skilled in the art. Suitable delivery methods include, for example, by injection, as drops, aerosolized and/or topically. Genetic vaccines of the present invention can be contained in an aqueous excipient (e.g., phosphate-buffered saline) alone or in a carrier (e.g., lipid-based vehicles).

A recombinant virus vaccine of the present invention includes a recombinant molecule of the present invention that is packaged in a viral coat and that can be expressed in an animal after administration. Preferably, the recombinant molecule is packaging- or replication-deficient and/or encodes an attenuated virus. A number of recombinant viruses can be used, including, but not limited to, those based on alphaviruses, poxviruses, adenoviruses, herpesviruses, picornaviruses, and retroviruses. Preferred recombinant virus vaccines are those based on alphaviruses (such as Sindbis virus), raccoon poxviruses, species-specific herpesviruses and species-specific poxviruses. Examples of methods to produce and use alphavirus recombinant virus vaccines are disclosed in PCT Publication No. WO 94/17813.

When administered to an animal, a recombinant virus vaccine of the present invention infects cells within the immunized animal and directs the production of a protective protein that is capable of protecting the animal from filariasis or dirofilariasis caused by filarial or dirofilarial nematodes, respectively. By way of illustration, a single dose of a recombinant virus vaccine of the present invention can be from about 1×10⁴ to about 1×10⁸ virus plaque forming units (pfu) per kilogram body weight of the animal. Administration protocols are similar to those described herein for protein-based vaccines, with subcutaneous, intramuscular, intranasal and oral as routes of administration.

A recombinant cell vaccine of the present invention includes recombinant cells of the present invention that express a protein of the present invention. Preferred recombinant cells for this aspect include Salmonella, E. coli, Listeria, Mycobacterium, S. frugiperda, yeast, (including Saccharomyces cerevisiae and Pichia pastoris), BHK, CV-1, myoblast G8, COS (e.g., COS-7), Vero, MDCK and CRFK recombinant cells. Recombinant cell vaccines of the present invention can be administered in a variety of ways but have the advantage that they can be administered orally, preferably at doses ranging from about 10⁸ to about 10¹² cells per kilogram body weight. Administration protocols are similar to those described herein for protein-based vaccines. Recombinant cell vaccines can include whole cells, cells stripped of cell walls or cell lysates.

In some aspects of the composition of the invention, all of the antigens are present in the composition in the same amount. In further aspects, the antigens are present in the composition in different amounts (i.e., at least one antigen is present in an amount that is different than one or more of the other antigens of the composition). By way of illustration a fusion protein may be composed of one copy of each of ALT2, TSP, and TPX2, and two copies of HSP.

Optimal amounts of components for a particular immunogenic composition can be ascertained by standard studies involving observation of appropriate immune responses in subjects. For example, in another aspect, the dosage for human vaccination is determined by extrapolation from animal studies to human data. In another aspect, the dosage is determined empirically.

As is known in the art, there are three groups of filarial nematodes, classified according to the niche within the body that they occupy: lymphatic filariasis, subcutaneous filariasis, and serous cavity filariasis. Lymphatic filariasis is caused by the worms W. bancrofti, B. malayi and B. timori. These worms occupy the lymphatic system, including the lymph nodes, and cause fever, lymphadenitis (swelling of the lymph nodes), lymphangitis (inflammation of the lymphatic vessels in response to infection), and lymphedema (elephantiasis). Subcutaneous filariasis may be caused by Loa loa (the African eye worm), Mansonella stretocerca, O. volvulus, Dracunculus medinensis, or Dirofilaria immitis. Many of these worms including Dirofilaria repens occupy the subcutaneous layer of the skin, in the fat layer, and present with skin rashes, urticarial papules, and arthritis, as well as hyper- and hypopigmentation macules. Onchocerca volvulus manifests itself in the eyes, causing “river blindness.” Adult Dirofilaria immitis reside in pulmonary arteries and in the heart and are the causal agent of heartworm disease. Serous cavity filariasis is caused by the worms M. perstans and M. ozzardi, which occupy the serous cavity of the abdomen. Serous cavity filariasis presents with symptoms similar to subcutaneous filariasis, in addition to abdominal pain, because these worms are also deep tissue dwellers.

Dogs infected with Brugia malayi develop clinical lymphedema, scrotal enlargement, conjunctivitis and lymphagitis similar to the human lymphatic filariasis; however, the pathology is not as severe as in the human. Since dogs carry the infection in the nature, humans can get the Brugia malayi infections from dogs. Thus, zoonotic infections are common in the endemic areas, where dogs and cats carry the infection in the nature and they transmit the infection to the humans. Dogs and cats can also be infected with Brugia malayi under laboratory conditions. Thus, an immunogenic composition developed against lymphatic filariasis in dogs are also important in blocking transmission of the disease in the human.

The efficacy of a multivalent immunogenic composition of the present invention to protect an animal from filariasis or dirofilariasis caused by filarial or dirofilarial nematodes can be tested in a variety of ways including, but not limited to, detection of protective antibodies (using, for example, proteins of the present invention), detection of cellular immunity within the treated animal, and/or challenge of the treated animal with the a filarial nematode to determine whether the treated animal is resistant to disease and fails to exhibit one or more signs of disease. Challenge studies can include implantation of chambers including filarial or dirofilarial nematode larvae into the treated animal and/or direct administration of larvae to the treated animal. In one aspect, therapeutic compositions can be tested in animal models such as mice, jirds (Meriones unguiculatus), mastomys (e.g., Mastomys natalensis) and/or dogs. Such techniques are known to those skilled in the art.

To detect the presence/amount of anti-filarial nematode antibodies, e.g., protective or neutralizing antibodies resulting from the vaccination of an animal, this invention also provides a method and kit for efficacy evaluation, as well as for detecting prior exposure to filarial proteins and/or infection with a filarial nematode. In accordance with such a method, one or more antigenic proteins/epitopes is contacted with a biological sample from an animal and binding between the antigenic proteins/epitopes and antibodies in the biological sample is quantitively or qualitatively determined as described herein, wherein the presence and/or amount of antibodies to the antigenic proteins/epitopes is indicative of vaccine efficacy, as well as prior exposure to filarial proteins or an existing infection with a filarial nematode. In certain aspects, the method and kit use an array-based format in which serial dilutions of one or more antigens or epitopes are printed. In some aspects, the one or more of the filarial nematode proteins are present on one or more solid surfaces or particles. In other aspects, the one or more of the filarial nematode proteins are in an array so that the presence of multiple antibodies can be assessed in a single assay due to the multiplexing capability of an array-based approach. In this respect, the array can contain one or more of ALT2, TSP, TPX2, or HSP protein or an epitope thereof. In other aspects, the array at least contains each of the proteins used in the multivalent immunogenic composition. For example, to assay for protective or neutralizing antibodies against a multivalent immunogenic composition containing HSP, ALT2, TPX2, and TSP, the array would contain HSP, ALT2, TPX2, and TSP, or a fusion protein thereof.

For testing for the presence of a filarial nematode, this invention also provides a method and kit for detecting a filarial nematode. The assay method generally includes the steps of contacting, in vitro, a biological sample with one or more binding agents against filarial nematode proteins selected from the group of ALT2, TSP, TPX2, and HSP or fragments thereof. The bound binding agents are then detected. The bound binding agents can be detected using automated detection of binding such as an image reader of an ELISA assay, and if a bound binding agent is detected, the data indicating that a bound binding agent has been detected can be transferred, e.g., to a computer display or on a paper print out. Detection of a filarial nematode protein indicates that the sample or subject from which the sample was obtained has filariasis. Therefore, detection allows selection of treatment options for the subject. Thus, in one aspect, if one or more of ALT2, TSP, TPX2, and HSP is detected, the patient will be given a treatment suitable for filariasis, including but not limited to treatment with diethylcarbamazine, mebendazole, flubendazole, albendazole, ivermectin or a combination thereof.

A biological sample is any material to be tested for the presence or amount of a protein of interest (e.g., an antibody or antigen/epitope). The sample can be a fluid sample, preferably a liquid sample. Examples of liquid samples that may be tested in accordance with this invention include bodily fluids including blood, serum, plasma, saliva, urine, ocular fluid, semen, and spinal fluid. Viscous liquid, semi-solid, or solid specimens (e.g., human tissue, or mosquito or fly tissue) may be used to create liquid solutions, eluates, suspensions, or extracts that can be samples. In some aspects, the biological sample is undiluted. In other aspects, the sample is diluted or concentrated depending on the detection application.

In certain aspects, one can concentrate the proteins in the sample by using a solid surface coated with a monoclonal antibody to capture the protein. The recovered captured proteins can then be analyzed using any suitable method described herein. The solid surface can be, e.g., beads, such as magnetic beads, polystyrene beads, or gold beads, or in an array or a microarray format using a glass, a plastic or a silicon chip. Such protein capture can be also a part of a channel in a microfluidic device.

Binding agents of use in this invention include an antibody, an antibody fragment, or an antibody derivative (e.g., an aptamer) which specifically binds to a cognate filarial nematode protein. Specific binding between two entities generally refers to an affinity of at least 10⁶, 10⁷, 10⁸, 10⁹, or 10¹⁰ M⁻¹. Affinities greater than 10⁸ M⁻¹ are desired to achieve specific binding.

When the binding agent is an antibody, the antibody can be produced by natural (i.e., immunization) or partial or wholly synthetic means. Antibodies can be monoclonal or polyclonal and include commercially available antibodies. An antibody can be a member of any immunoglobulin class, including any of the human classes: IgG, IgM, IgA, IgD, and IgE. Bispecific and chimeric antibodies are also encompassed within the scope of the present invention. Derivatives of the IgG class, however, are desirable. Further, an antibody can be of human, mouse, rat, goat, sheep, rabbit, chicken, camel, or donkey origin or other species which may be used to produce native or human antibodies (i.e., recombinant bacteria, baculovirus or plants).

For example, naturally-produced monoclonal antibodies can be generated using classical cloning and cell fusion techniques or techniques wherein B-cells are captured and nucleic acids encoding a specific antibody are amplified (see, e.g., US 2006/0051348). In such methods, a collection of proteins or an individual protein (e.g., a peptide or polypeptide) can be used for the initial immunization and in the context of antibody production is referred to herein as the antigen. The antigen of interest is typically administered (e.g., intraperitoneal injection) to wild-type or inbred mice (e.g., BALB/c) or rats, rabbits, chickens, sheep, goats, or other animal species which can produce native or human antibodies. The antigen can be administered alone, or mixed with an adjuvant. After the animal is boosted, for example, two or more times, the spleen or large lymph node, such as the popliteal in rat, is removed and splenocytes or lymphocytes are isolated and fused with myeloma cells using well-known processes, for example, see Kohler & Milstein ((1975) Nature 256:495-497) or Harlow & Lane (Antibodies: A Laboratory Manual (Cold Spring Harbor Laboratory, New York (1988)). The resulting hybrid cells are then cloned in the conventional manner, e.g., using limiting dilution, and the resulting clones, which produce the desired monoclonal antibodies, are cultured (see Stewart (2001) Monoclonal Antibody Production. In: Basic Methods in Antibody Production and Characterization, Howard and Bethell (eds.), CRC Press, Boca Raton, FL, pp. 51-67).

Alternatively, antibodies can be derived by a phage display method. Methods of producing phage display antibodies are known in the art, e.g., see Huse, et al. ((1989) Science 246(4935):1275-81). Selection of antibodies is based on binding affinity to a protein or proteins of interest.

An antibody fragment encompasses at least a significant portion of the full-length antibody's specific binding ability. Examples of antibody fragments include, but are not limited to, Fab, Fab′, F(ab′)₂, scFv, Fv, dsFv, diabody, Fd fragments or microbodies. An antibody fragment can contain multiple chains which are linked together, for instance, by disulfide linkages. A fragment can also optionally be a multi-molecular complex. A functional antibody fragment will typically include at least about 50 amino acid residues and more typically will include at least about 200 amino acid residues. The antibody fragment can be produced by any means. For instance, the antibody fragment can be enzymatically or chemically produced by fragmentation of an intact antibody or it can be recombinantly-produced from a gene encoding the partial antibody sequence. Alternatively, the antibody fragment can be wholly or partially synthetically-produced.

Peptide aptamers which specifically bind to a protein are, in general, rationally designed or screened for in a library of aptamers (e.g., provided by Aptanomics SA, Lyon, France). In general, peptide aptamers are synthetic recognition molecules whose design is based on the structure of antibodies. Peptide aptamers are composed of a variable peptide loop attached at both ends to a protein scaffold. This double structural constraint greatly increases the binding affinity of the peptide aptamer to levels comparable to that of an antibody (nanomolar range).

Recombinant production of binding agents of this invention can be achieved using conventional molecular biology techniques and commercially available expression systems. Furthermore, binding agents can be produced using solid-phase techniques (see, e.g., Merrifield (1963) J. Am. Chem. Soc. 85:2149-2154; Seeberger (2003) Chem. Commun. (Camb) (10):1115-21). Protein synthesis can be performed using manual techniques or by automation. Automated synthesis can be achieved, for example, using Applied Biosystems 431A Peptide Synthesizer (Perkin Elmer, Boston, MA). Various fragments of a binding agent can be chemically-synthesized separately and combined using chemical methods to produce a full-length molecule.

Moreover, combinatorial chemistry approaches can be used to produce binding agents (see, e.g., Lenssen, et al. (2002) Chembiochem. 3(9):852-8; Khersonsky, et al. (2003) Curr. Top. Med. Chem. 3(6):617-43; Anthony-Cahill & Magliery (2002) Curr. Pharm. Biotechnol. 3(4):299-315).

The binding agents described herein can be labeled. In some aspects, the binding agent is an antibody labeled by covalently linking the antibody to a direct or indirect label. A direct label can be defined as an entity, which in its natural state, is visible either to the naked eye or with the aid of an optical filter and/or applied stimulation, e.g., ultraviolet light, to promote fluorescence. Examples of colored labels which can be used include metallic sol particles, gold sol particles, dye sol particles, dyed latex particles or dyes encapsulated in liposomes. Other direct labels include radionuclides and fluorescent or luminescent moieties.

Indirect labels such as enzymes can also be used according to the invention. Various enzymes are known for use as labels such as, for example, alkaline phosphatase, horseradish peroxidase, lysozyme, glucose-6-phosphate dehydrogenase, lactate dehydrogenase and urease. For a detailed discussion of enzymes in immunoassays see Engvall (1980) Methods of Enzymology 70:419-439.

The proteins described herein (i.e., antibodies or antigens/epitopes) can be attached to a surface. Examples of useful surfaces on which the protein can be attached for diagnostic purposes include nitrocellulose, PVDF, polystyrene, nylon or other suitable plastic. The surface or support may also be a porous support (see, e.g., U.S. Pat. No. 7,939,342).

Further, the proteins of the invention can be attached to a particle or bead. For example, antibodies to the filarial nematode proteins or the filarial nematode proteins themselves can be conjugated to superparamagnetic microparticles, e.g., as used in LUMINEX-based multiplex assays.

The filarial nematode proteins of this invention may be isolated and/or purified or produced synthetically or using recombinant nucleic acid technology. The purification may be partial or substantial. With reference to filarial nematode protein fragments, the term “fragment” refers to a protein having an amino acid sequence shorter than that of the proteins described herein. Preferably, such fragments are at least 5 consecutive amino acids long or up to 35 amino acids long. In certain aspects, the protein fragment includes at least one epitope. An “epitope” is a feature of a molecule, such as primary, secondary and/or tertiary peptide structure, and/or charge, that forms a site recognized by an immunoglobulin, T cell receptor or HLA molecule. Alternatively, an epitope can be defined as a set of amino acid residues which is involved in recognition by a particular immunoglobulin, or in the context of T cells, those residues necessary for recognition by T cell receptor proteins and/or Major Histocompatibility Complex (MHC) receptors.

The fragments of the invention can be isolated, purified or otherwise prepared/derived by human or non-human means. For example, epitopes can be prepared by isolating the filarial nematode protein fragment from a bacterial culture, or they can be synthesized in accordance with standard protocols in the art. Synthetic epitopes can also be prepared from amino acid mimetics, such as D isomers of natural occurring L amino acids or non-natural amino acids such as cyclohexylalanine.

In some aspects, the filarial nematode protein or protein fragment is conjugated or fused to a high molecular weight protein carrier to facilitate antibody production. In some aspects, the high molecular weight protein is bovine serum albumin, thyroglobulin, ovalbumin, fibrinogen, or keyhole limpet hemocyanin. A particularly preferred carrier is keyhole limpet hemocyanin.

Any suitable immunoassay method may be used, including those which are commercially, available, to determine the level of at least one of the specific filarial nematode proteins, protein fragments or protective/neutralizing antibodies according to the invention. Extensive discussion of the known immunoassay techniques is not required here since these are known to those of skill in the art. Typical suitable immunoassay techniques include sandwich enzyme-linked immunoassays (ELISA), radioimmunoassays (RIA), competitive binding assays, homogeneous assays, heterogeneous assays, etc. Various of the known immunoassay methods are reviewed, e.g., in Methods in Enzymology (1980) 70:30-70 and 166-198.

In some aspects, the immunoassay method or assay includes a double antibody technique for measuring the level of the filarial nematode proteins or protein fragments in the biological sample. According to this method one of the antibodies is a “capture” antibody and the other is a “detector” antibody. The capture antibody is immobilized on a solid support which may be any of various types which are known in the art such as, for example, microtiter plate wells, beads, tubes and porous materials such as nylon, glass fibers and other polymeric materials. In this method, a solid support, e.g., microtiter plate wells, coated with a capture antibody, preferably monoclonal, raised against the particular protein of interest, constitutes the solid phase. The biological sample, which may be diluted or not, typically at least 1, 2, 3, 4, 5, 10, or more standards and controls are added to separate solid supports and incubated. When the protein of interest is present in the sample it is captured by the immobilized antibody which is specific for the protein in question. After incubation and washing, a detector antibody, e.g., a polyclonal rabbit anti-marker protein antibody, is added to the solid support. The detector antibody binds to the protein bound to the capture antibody to form a sandwich structure. After incubation and washing an anti-IgG antibody, e.g., a polyclonal goat anti-rabbit IgG antibody, labeled with an enzyme such as horseradish peroxidase (HRP) is added to the solid support. After incubation and washing a substrate for the enzyme is added to the solid support followed by incubation and the addition of an acid solution to stop the enzymatic reaction.

The degree of enzymatic activity of immobilized enzyme is determined by measuring the optical density of the oxidized enzymatic product on the solid support at the appropriate wavelength, e.g., 450 nm for HRP. The absorbance at the wavelength is proportional to the amount of protein of interest in the sample. A set of marker protein standards is used to prepare a standard curve of absorbance vs. filarial nematode protein concentration. This method is useful because test results can be provided in 45 to 50 minutes and the method is both sensitive over the concentration range of interest for each filarial nematode protein and is highly specific.

The standards may be positive samples containing various concentrations of the protein to be detected to ensure that the reagents and conditions work properly for each assay. The standards also typically include a negative control, e.g., for detection of contaminants. In some aspects of the aspects of the invention, the positive controls may be titrated to different concentrations, including non-detectable amounts and clearly detectable amounts, and in some aspects, also including a sample that shows a signal at the threshold level of detection in the biological sample.

The method of the invention can be carried out in various assay device formats including those described in U.S. Pat. Nos. 6,426,050, 5,910,287, 6,229,603, and U.S. Pat. No. 6,232,114 to Aurora Biosciences Corporation. The assay devices used according to the invention can be arranged to provide a quantitative or a qualitative (present/not present) result. In some aspects, the method includes the use of a microtiter plate or a microfluidic device format. The assays may also be carried out in automated immunoassay analyzers which are known in the art and which can carry out assays on a number of different samples. These automated analyzers include continuous/random access types. Examples of such systems are described in U.S. Pat. Nos. 5,207,987, 5,518,688, 6,448,089, and 6,814,933. Various automated analyzers that are commercially available include the OPUS® and OPUS MAGNUM® analyzers.

Another assay format which can be used according to the invention is a rapid manual test which can be administered at the point-of-care at any location. Typically, such point-of-care assay devices will provide a result which is either “positive,” i.e., showing the protein is present, or “negative” showing that the protein is absent. Typically, a control showing that the reagents worked in general is included with such point-of-care system. Point-of-care systems, assays and devices have been well described for other purposes, such as pregnancy detection (see, e.g., U.S. Pat. Nos. 7,569,397 and 7,959,875). Accordingly, the invention also provides devices, such as point-of-care test strips and microfluidic devices to perform the in vitro assays of the present invention.

It should be recognized also that the assay devices used according to the invention can be provided to carry out one single assay for a particular protein or to carry out a plurality of assays, from a single volume of body fluid, for a corresponding number of different filarial nematode proteins or antibodies thereto. In some aspects, an assay device of the latter type is one which can provide a semi-quantitative result for the filarial nematode protein or antibodies measured according to the invention, i.e., one or more of ALT2, TSP, TPX2, and HSP, or antibodies thereto. These devices typically are adapted to provide a distinct visually detectable colored band at the location where the particular protein of interest is located when the concentration of the protein is above the threshold level. For additional detailed discussion of assay types which can be utilized according to the invention as well as various assay formats and automated analyzer apparatus see, e.g., U.S. Pat. No. 5,747,274. Filarial nematode protein detection can further be performed using multiplex technologies.

In other aspects, the assays or immunoassays of the invention include beads coated with a binding agent against a filarial nematode protein or a fragment thereof, or antibody. Commonly used are polystyrene beads that can be labeled to establish a unique identity. Detection is performed by flow cytometry. Other types of bead-based immunoassays are known in the art, e.g., laser bead immunoassays and related magnetic bead assays (see, e.g., Fritzler, et al. (2009) Expert Opinion on Medical Diagnostics 3:81-89).

The methods of the invention can be automated using robotics and computer directed systems. The biological sample can be injected into a system, such as a microfluidic devise entirely run by a robotic station from sample input to output of the result. The step of displaying the result can also be automated and connected to the same system or in a remote system. Thus, the sample analysis can be performed in one location and the result analysis in another location, the only connection being, e.g., an internet connection, wherein the analysis is subsequently displayed in a format suitable for either reading by a health professional or by a patient.

In certain aspects, the presence of any one or any combination of protective/neutralizing antibodies described herein identifies a subject as having been immunized with a multivalent immunogenic composition against a filarial nematode. Thus, depending on antibody titer, the subject may or may not receive additional booster vaccinations.

In some aspects, the presence of any one or any combination of the filarial nematode proteins described herein identifies a subject as having a filarial nematode infection. Thus, the subject is diagnosed as having filariasis and, in certain aspects of this invention, treated with diethylcarbamazine, mebendazole, flubendazole, albendazole, ivermectin or a combination thereof. In one aspect, the diagnosis can be made if the presence of any one of the filarial nematode proteins is detected in the subject's sample. In another aspect, treatment is prescribed or administered if at least two of the filarial nematode proteins are identified positively in the biological sample.

Kits provided according to this invention include one or more binding agents, e.g., antibodies or antibody fragments, or filarial nematode proteins, and optionally a device with a solid surface. In some aspects, the solid surface is a bead, slide, assay plate (e.g., a multiwell plate) or a lateral flow device, to which the binding agents/proteins are bound. In some aspects, the kit further includes one or more standards or controls.

In some aspects, the invention provides a microplate-based array for multiplex immunoassays. In accordance with some aspects, each well can contain a single antibody against at least one of the listed filarial nematode proteins. In other aspects, each well contains an array of antibodies against at least two or more of the listed filarial nematode proteins. In certain aspects, each well of the plate includes an antibody to two, three, four, or five of the following proteins: ALT2, TSP, TPX2, and HSP. In particular aspects, each well of the plate includes an antibody to each of ALT2, TSP, TPX2, and HSP.

In other aspects, each well contains an array of at least two or more of the filarial nematode proteins of this invention. In certain aspects, each well of the plate includes two, three, or four of the following proteins: ALT2, TSP, TPX2, and HSP. In particular aspects, each well of the plate includes each of ALT2, TSP, TPX2, and HSP.

In other aspects, the invention provides simple to use point-of-care diagnostic test strips akin to pregnancy detection strips, wherein the strip includes at least one antibody against at least one of the listed filarial nematode proteins. In alternative aspects, the invention provides simple to use point-of-care diagnostic test strips, wherein the strip includes at least one of the instant filarial nematode proteins.

The test strip may include a positive and negative control to show the user that the reagents work properly and/or that the sample has been added to the strip properly. The strips may be provided with or without a casing and with or without additional reagents. Diagnostic test strips for lateral flow assays, such as the test strip assay described herein, may be constructed as described in the art, see, e.g., US 2010/0196200; US 2010/0129935; US 2009/0253119; and US 2009/0111171. Suitable materials for test strips include, but are not limited to, materials derived from cellulose, such as filter paper, chromatographic paper, nitrocellulose, and cellulose acetate, as well as materials made of glass fibers, nylon, dacron, PVC, polyacrylamide, cross-linked dextran, agarose, polyacrylate, ceramic materials, and the like. The material or materials of the test strip may optionally be treated to modify their capillary flow characteristics or the characteristics of the applied sample. For example, the sample application region of the test strip may be treated with buffers to correct the pH or specific gravity of an applied sample, to ensure optimal test conditions.

The invention is described in greater detail by the following non-limiting examples.

### Example 1: BmHAXT Fusion Constructs

BmHAXT (his-tag) and BmHAXT (tag-free). To ensure traceability from clone development throughout process development, all cloning, expression, and purification reagents were carefully sourced and documented to ensure the absence of animal products. The reference N-terminal 6×His-tagged BmHAXT fusion, referred to as “BmHAXT (his-tag),” has nucleotide and polypeptide sequences as set forth in SEQ ID NO:16 and SEQ ID NO:9, respectively. Removal of the 6×His tag from the BmHAXT (his-tag) fusion resulted in a 1557 nucleotide sequence, which was codon optimized for expression in E. coli K12. The untagged BmHAXT fusion, referred to as “BmHAXT (tag-free),” has nucleotide and polypeptide sequences as set forth in SEQ ID NO:20 and SEQ ID NO:21, respectively.

BmHAXT (ΔCys). Upon recombinant expression of BmHAXT (tag-free), it was observed that the fusion protein rapidly formed large aggregates. To address this, a third generation fusion protein, referred to as “BmHAXT (ΔCys),” was designed in silico by changing all cysteine residues to serine residues using the codon optimized sequence of BmHAXT (tag-free) as a template. There are a total of 17 cysteine residues in BmHAXT (tag-free) interspersed amongst the four proteins (Table 2). For the mutagenesis, cysteine codons with the nucleotide sequence TOO were mutated to AGO serine codons by changing the thymine base in the first position to an adenine base (Table 2). Cysteine codons with the nucleotide sequence TGT were mutated to TCT serine codons by changing the guanine base in the second position to a cytosine base (Table 2).

TABLE 2

| | | Cys codon in | Ser codon in |
|---|---|---|---|
| Mut# | AA# | BmHAXT (tag-free) | BmHAXT (ΔCys) |
| 1 | C64S | TGC | AGC |
| 2 | C132S | TGT | TCT |
| 3 | C179S | TGT | TCT |
| 4 | C185S | TGC | AGC |
| 5 | C195S | TGT | TCT |
| 6 | C208S | TGT | TCT |
| 7 | C210S | TGT | TCT |
| 8 | C217S | TGC | AGC |
| 9 | C232S | TGT | TCT |
| 10 | C240S | TGC | AGC |
| 11 | C294S | TGC | AGC |
| 12 | C318S | TGT | TCT |
| 13 | C415S | TGT | TCT |
| 14 | C477S | TGT | TCT |
| 15 | C478S | TGC | AGC |
| 16 | C494S | TGT | TCT |
| 17 | C504S | TGC | AGC |

It was anticipated that cysteine residues were not required to achieve comparable immunogenicity and that removal of cysteines would reduce disulfide bond formation thereby decreasing aggregation due at least in part to aberrant disulfide bonds forming within and between the four individual proteins during the refolding process.

The redesigned BmHAXT (ΔCys) gene was produced as a gene fragment from Integrated DNA Technologies (IDT, Coralville, IA). The nucleotide and polypeptide sequences of BmHAXT (ΔCys) are set forth in SEQ ID NO:17 and SEQ ID NO:10, respectively. The BmHAXT (ΔCys) was cloned into the pET29a(+) expression vector (Millipore Sigma, Burlington, MA). Plasmids were transformed into E. coli Turbo cells (NEB, Ipswich, MA) and transformants selected on Luria broth (LB) plates supplemented with 50 μg/mL of kanamycin sulfate (LB-Kan). Transformants were screened by polymerase chain reaction (PCR) for correct sized inserts and presumptive positives sequence were confirmed. Plasmid from a single confirmed clone was transformed into the commercial E. coli expression strain HMS174 (DE3) and transformants selected on LB-Kan plates. Resulting colonies were screened for expression of a ˜60 kDa band corresponding to the predicted size of BmHAXT (ΔCys).

Cell Banking. A research cell bank of 162 vials of E. coli HMS174 (DE3) strain containing plasmid BmHAXT ΔCys clone 6B in vector pET29a was laid down. The clone was grown in animal-free LB-Kan to an OD₆₀₀ of ˜1.0. After confirming the culture purity by microscopic observation and growth on both selective and non-selective media, the culture was mixed 1:1 with sterile LB-Kan broth containing 20% plant-derived glycerol. The cell bank was stored at −80° C. Confirmation of expression before and after cell banking was performed and the expressed protein was confirmed to be localized to the insoluble fraction (IBs or inclusion bodies).

Fermentation. Fermentation was optimized at the two-liter scale and a fermentation batch record was developed. Briefly, a cell bank vial was inoculated into two 200 mL of LB-Kan broth and grown to a cell density of >3 OD₆₀₀. Protein expression was induced by addition of isopropyl β-D-1-thiogalactopyranoside (IPTG) to a final concentration of 1 mM. Agitation-induced foaming was minimized by constant addition of Antifoam 204 (Sigma, St. Louis, MO) added to a final concentration of 0.01% throughout the growth and induction phase. Fermentation was performed at 37±1° C., with air flow at 30 L/min, and pH maintained at 7.0±0.2 by addition of 6 N NH₄OH (base) or 5 N HCl (acid) as needed. Dissolved oxygen was held at a minimum of 40% by cascading with agitation followed by oxygen supplementation. Harvest by centrifugation was performed 3 hours post-induction.

Process Development. The process for purifying BmHAXT (ΔCys) was essentially identical to the process used to purify BmHAXT (tag-free) with two key differences. The first was the omission of dithiothreitol in all BmHAXT (ΔCys) purification steps, due to the deletion of cysteine residues and therefore disulfide bonds. The second difference was reducing the washing stringency (NaCl concentration reduced from 180 mM to 160 mM) during the anion exchange purification washing steps using a Q SEPHAROSE® (QFF) column.

Isolation of IBs. E. coli cell pellets were thawed and resuspended in 5 mL lysis buffer (50 mM tris and 0.5% TRITON™ X-100 pH 8.0) per gram of wet cell paste and mixed by vortexing then pipetting until no visible clumps were observed. The suspension was passed three times through a LM10 microfluidizer (Microfluidics Corp., Westwood, MA) at 15,000 psi allowing for intermittent cooling between passes. The IB fraction was pelleted by centrifugation at 14,000×g for 30 minutes. The IB pellet was resuspended in mL of 1% 3-[(3-cholamidopropyl)dimethylammonio]-1-propanesulfonate (CHAPS) detergent solution per gram IB and then pelleted by centrifugation as above. The IB pellet was resuspended in 20 mL 25% isopropyl alcohol per gram of TB and pelleted again as above. Washed IB pellet was resuspended in 20 mL of solubilization buffer (50 mM tris, 8 M urea pH 8.0) per gram of IB and rolled gently at 4° C. for 16-20 hours. The solubilized crude IB solution was clarified by centrifugation at 15,000×g for 3 hours at 4° C. The supernatant containing the solubilized BmHAXT (ΔCys) was decanted to a fresh container and stored at −80° C. until purification.

CAPTO™ Q ImpRes Ion Exchange Chromatography. Solubilized IB solution (180 mL) was passed across a strong anion exchange resin, CAPTO™ Q ImpRes (Cytiva, Marlborough, MA), at a flow rate of 10 mL/min. The resin was washed to baseline with 4-5 column volumes of Q Wash Buffer (50 mM tris, 8 M urea, 160 mM NaCl, 10 mM DTT pH 8.0). Protein was eluted to baseline using 1-2 column volumes of Q Elution Buffer (50 mM tris, 8 M urea, 300 mM NaCl pH 8.0). The emerging peak was analyzed by SDS-PAGE to confirm enrichment of BmHAXT (ΔCys) protein.

CAPTO™ SP ImpRes Ion Exchange Chromatography. The eluted protein pool was diluted 1:8 with SP loading buffer (20 mM acetate, 8 M urea, 10 mM DTT pH 4.0). The adjusted solution was passed across the strong cation exchange resin, CAPTO™ SP ImpRes (Cytiva), at a flow rate of 4 mL/min. The resin was washed to baseline with 3-5 column volumes of SP Wash Buffer (20 mM acetate, 8 M urea, 300 mM NaCl pH 4.0) to remove non-specifically bound contaminant proteins. Protein was eluted to baseline with 1.5-2 column volumes of SP elution buffer (20 mM acetate, 8 M urea, 1 M NaCl pH 4.0). The emerging peak was analyzed by SDS-PAGE to confirm enrichment of BmHAXT (ΔCys) protein.

Diafiltration. Pooled protein was buffer-exchanged into 20 volumes of 50 mM Tris pH 8.0 by tangential flow filtration using PELLICON® 10 kDa molecular weight cut-off diafiltration cartridges (Millipore Sigma) and volume was reduced until the protein concentration by OD₂₈₀ absorbance was 2-4 mg/mL. Glycerol was added to 5% (v/v) and the solution was sterile-filtered through 0.2 μM filters and then adjusted to a final concentration of 1 mg/mL.

Triplicate 1 μg loads of BmHAXT (ΔCys) and bovine serum albumin (BSA) standards were analyzed by reducing SDS-PAGE and quantified by ImageJ densitometry analysis to confirm both concentration and purity. Identity was confirmed by western blot analysis with monkey anti-BmHAXT (his-tagged) antisera at 1/10,000 and detected with a 1/10,000 dilution of horseradish peroxidase (HRP)-conjugated goat anti-monkey IgG (H+L) secondary antibody (Thermo Fisher, Rockford, IL). Presence of E. coli host cell proteins was detected by western blot analysis using a 1/1,000 dilution of rabbit anti-E. coli Host Cell Protein (HCP) polyclonal antibody (Rockland Immunochemicals, Inc., Limerick, PA) detected with a 1/2,000 dilution of HRP-conjugated donkey anti-rabbit IgG (H+L) secondary antibody (Southern Biotech, Birmingham, AL). Residual endotoxin was measured using Limulus amebocyte lysate (LAL) assay (Charles River Laboratories, Worcester, MA).

Additional BmHAXT Variants. Two additional mutant proteins including 12 amino acid flexible glycine-serine (GS) linkers (Gly-Gly-Gly-Ser-Gly-Gly-Gly-Ser-Gly-Gly-Gly-Ser; SEQ ID NO:28) inserted between each of the four proteins in the fusion were generated and termed “BmHAXT (GS)” and “BmHAXT (ΔCys+GS).” The nucleotide and polypeptide sequences of BmHAXT (GS) are set forth in SEQ ID NO:18 and SEQ ID NO:11, respectively. The nucleotide and polypeptide sequences of BmHAXT (ΔCys+GS) are set forth in SEQ ID NO:19 and SEQ ID NO:12, respectively. To prevent recombination of the genes due to the presence of the identical linker sequences, the nucleotide sequence encoding the three glycine linkers were designed differently by randomizing the codons for the 12 amino acids (Table 3).

TABLE 3

| Nucleotide Sequences Encoding | SEQ ID |
|---|---|
| GGGSGGGSGGGS (SEQ ID NO: 28) Linker | NO: |
| ggcggcggtagcggcggtggctctggcggtggttcc | 23 |
| ggtggcggttctggtggcggctccggtggtggcagc | 24 |
| ggtggtggctccggtggcggtagcggcggcggttct | 25 |

All 17 cysteines in BmHAXT (ΔCys+GS) were mutated to serines using the same codon substitutions as described in Table 3 except the positions of some of these mutations were different to reflect the insertion of the glycine-serine linkers. The intended purpose of the linkers was to promote native folding of each individual protein. The nucleotide and polypeptide sequences of BmHAXT (GS) are set forth in SEQ ID NO:18 and SEQ ID NO:11, respectively. BmHAXT (ΔCys+GS) was identical to BmHAXT (GS) plus all 17 cysteine residues were deleted. The nucleotide and polypeptide sequences of BmHAXT (ΔCys+GS) are set forth in SEQ ID NO:19 and SEQ ID NO:12, respectively. Like BmHAXT (QCys), both BmHAXT (GS) and BmHAXT (ΔCys+GS) were designed in silico and produced as GeneBlocks from IDT. Cloning of both genes was exactly as described for BmHAXT (ΔCys). The BmHAXT (GS) was purified using the exact procedure described for BmHAXT (tag-free). The BmHAXT (ΔCys+GS) was purified using the exact procedure described for BmHAXT (ΔCys). With respect to purity of the four proteins, it was found that the cysteine-deleted mutants were significantly purer with respect to the primary band at 65 kDa. The BmHAXT (ΔCys) and BmHAXT (ΔCys+GS) proteins seemed to be significantly improved particularly with respect to degradation bands in the 16-50 kDa region.

Stability Assays. A short-term stability assay (Table 4) was performed to compare the BmHAXT (tag-free) with the three variants: BmHAXT (ΔCys), BmHAXT (GS), and BmHAXT (ΔCys+GS). Notably, both of the mutant variants containing the GS linker aggregated as much or more that the wild-type fusion protein and were produced at lower levels. Additionally, the GS-containing mutants appeared to be less protective in mouse studies. Therefore, the immunogenicity and stability data resulted in both of these mutants being eliminated for further consideration.

TABLE 4

| | | |
|---|---|---|
| Temperatures (5) | Timepoints (7) | Readouts (3) |

| | | | | |
|---|---|---|---|---|
| −80° | C.* | Day | 0* | SDS-PAGE |
| −20° | C. | Day | 3 | |
| 4-8° | C. | Week | 1 | Concentration by A₂₈₀ |
| 25° | C. | Week | 2 | |
| 42° | C. | Week | 3 | Dynamic light scattering |
| | | Week | 4 | |
| | | Week | 6 | |

*Reference

The stability study was performed as detailed in Table 4 by diluting each of four proteins to 0.5 mg/mL in 20 mM Tris pH 8.0. Approximately 40 aliquots of 0.1 mL of each protein were placed at the five temperatures. Protein stored at −80° C. in a Revco Ultra Low freezer was used as a control condition given that there should be no change in aggregation or degradation at this temperature. Storage at −20° C. was in a standard freezer (not frost-free). Storage at 4-8° C. was used to simulate a typical refrigerator. The 25° C. temperature simulated typical room-temperature conditions. Finally, 42° C. was used to simulate forced (accelerated) degradation and was meant to stress test the proteins. At each of the seven time points, one aliquot of each of the four proteins was removed from each of the five storage conditions and 1 μg total protein was resolved by reducing SDS-PAGE gel analysis and staining with SimplySafe stain.

Addition of GS linkers appeared to promote aggregation which was observable after 3 days and more pronounced at 25° C. and 42° C. Moreover, the aggregates also seemed to be slightly larger (˜300-400 kDa) than what was observed for BmHAXT (tag-free).

After 2 weeks, the BmHAXT (tag-free) and BmHAXT (GS) were nearly completely aggregated at 42° C. whereas, more than half of BmHAXT (ΔCys) and BmHAXT (ΔCys+GS) still remained as monomers. The results after 3 weeks revealed that all four proteins were fully aggregated at 42° C., with BmHAXT (GS) being the most aggregated. At the final time point of 6 weeks, BmHAXT (ΔCys) appeared to be the most stable with >90% of the protein remaining even at 25° C. It was concluded that the removal of cysteines greatly reduced aggregation and improved overall purity (with and without the GS linker).

### Example 2: Immunogenicity and Vaccine Efficacy of BmHAXT Fusion Proteins in Mice

Vaccine Proteins. BmHAXT (tag-free), BmHAXT (ΔCys), BmHAXT (GS), and BmHAXT (ΔCys+GS) proteins were expressed as recombinant proteins and purified as described in Example 1. The major focus of these animal studies was to determine whether mutating all cysteine residues in BmHAXT with serine residues, adding a linker glycine serine sequence (GS) in between the component proteins in the BmHAXT proteins, or combining cysteine mutation with GS linker in BmHAXT could increase the immunogenicity and vaccine efficacy of the redesigned vaccine. In these studies, glucopyranosyl lipid adjuvant (GLA) plus alum (GLA/Alum) was used as the adjuvant.

Animals and Parasite. Six- to eight-week-old male BALB/c mice purchased from Taconic Biosciences (Hudson, NY, USA) were housed at the University of Illinois College of Medicine Rockford animal facility. Use of animal in this study was approved by the animal care committee of the University of Illinois, Rockford following the National Institutes of Health guidelines for the care and use of laboratory animals. The infective larval stage (L3) of B. malayi was obtained from the NIAID/NIH Filariasis Research Reagent Resource Center (University of Georgia, Athens, GA, USA).

Vaccination Protocol. Twenty-five (25) mice were divided into five (5) groups of five mice each (Table 5). Group 1 mice received 1 μg of the adjuvant (GLA/Alum) in 100 μL of phosphate-buffered saline (PBS). Group 2 mice received 25 μg of BmHAXT (tag-free) plus 1 μg of GLA/Alum adjuvant on day 0, day 14 and day 28. Group 3 mice received 25 μg of BmHAXT (ΔCys) plus 1 μg of GLA/Alum adjuvant on day 0, day 14 and day 28. Group 4 mice received 25 μg of BmHAXT (GS) plus 1 μg of GLA/Alum adjuvant on day 0, day 14 and day 28. Group 5 mice received 25 μg of BmHAXT (ΔCys+GS) plus 1 μg of GLA/Alum adjuvant on day 0, day 14 and day 28. All injections were given subcutaneous (s.c.) into the right flank region of each mouse.

TABLE 5

| | | | | | Total |
|---|---|---|---|---|---|
| Group | N¹ | Sex | Antigen Dose/Mouse | Adjuvant Dose | Volume |
| 1 | 5 | M | 0 μg² | 1 μg GLA/Alum | 100 μL |
| 2 | 5 | M | 25 μg BmHAXT(tag-free) | 1 μg GLA/Alum | 100 μL |
| 3 | 5 | M | 25 μg BmHAXT(ΔCys) | 1 μg GLA/Alum | 100 μL |
| 4 | 5 | M | 25 μg BmHAXT(GS) | 1 μg GLA/Alum | 100 μL |
| 5 | 5 | M | 25 μg BmHAXT(ΔCys + GS) | 1 μg GLA/Alum | 100 μL |

¹Total N = 25 mice.
²PBS control.

Collection of Blood. Approximately 100 μL of whole blood was collected from the submandibular vein of each mouse on day 0 (pre-immune), day 14 (before first booster), day 28 (before second booster) and on day 48 (before challenge). Mice were anesthetized with a ketamine/xylazine formulation (0-100 mg/kg ketamine/xylazine 5-10 mg/kg) before collecting the blood. Serum samples were prepared and stored at −80° C. for serological analysis.

Titer of Antigen-Specific IgG Antibodies. The titer of BmHAXT-specific IgG antibodies in the sera samples were evaluated using an indirect ELISA as described previously (Chauhan et al. (2018) Front. Immunol. 9:1-11). Briefly, wells of a 96-well plate were coated overnight at 4° C. with 1 μg/mL of his-tagged BmHAXT. After washing the plates with PBS containing polysorbate 20 (PBST), the wells were blocked with 3% BSA. Following this, diluted (1:100, 1:1,000, 1:5,000, 1:10,000, 1:20,000, and 1:40,000) sera samples were added and incubated for 1 hour at room temperature. Following incubation, the plates were washed with PBST, and HRP-conjugated chicken anti-mouse IgG antibody (Thermo Fisher Scientific) was added as the secondary antibodies. Following a 1 hour incubation at room temperature, plates were washed with three rounds of PBST and distilled water before adding 1-STEP™ Ultra TMB-ELISA substrate (Thermo Fisher Scientific) to develop the color. The reaction was stopped using 0.16 M H₂SO₄, and optical density was determined at 450 nm in a BioTek Synergy 2 ELISA reader.

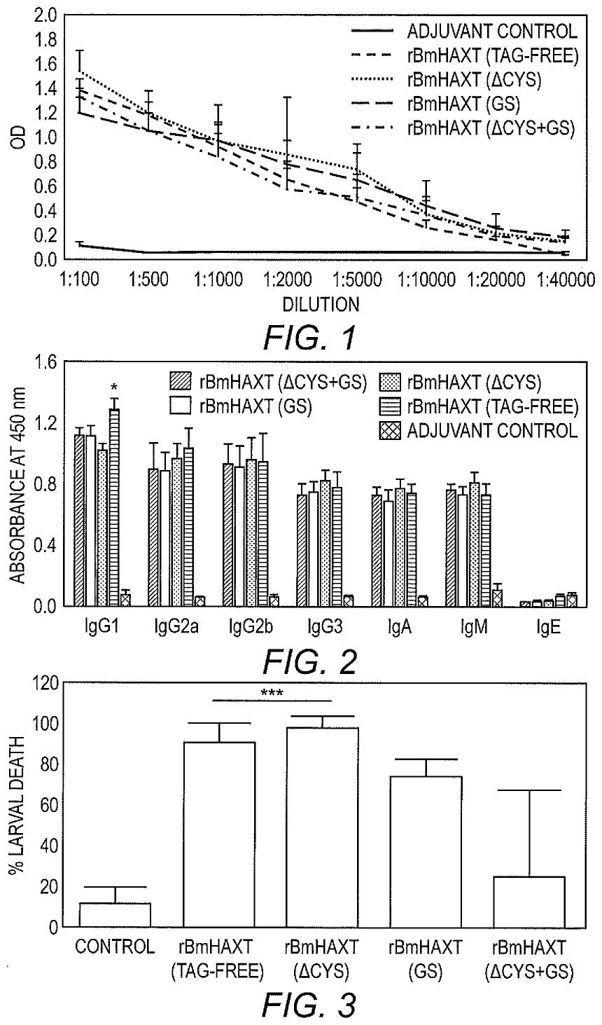

The results showed that compared to control mice that were given adjuvant alone, all mice vaccinated with BmHAXT (tag-free) or BmHAXT (ΔCys) or BmHAXT (GS) or BmHAXT (ΔCys+GS) showed significantly (p<0005) high titers of BmHAXT-specific IgG antibodies in the sera of mice confirming that ΔCys or GS or their combined modification did not alter the immunogenicity of the BmHAXT vaccine antigen (FIG. 1). These studies also indicated that the immune epitopes of the BmHAXT vaccine antigen were not altered despite the cysteine mutation and addition of GS linker sequences.

At 1:5,000 dilutions, titer of IgG antibodies were significantly (p<0.05) high in the sera of BmHAXT (ΔCys) immunized mice compared to BmHAXT (tag-free) immunized mice. IgG antibodies were detectable even at 1:20,000 dilutions of the sera samples in both BmHAXT (tag-free) and BmHAXT (ΔCys) immunized mice. These findings thus confirmed that redesigned BmHAXT (ΔCys) was immunogenic and the generated IgG antibodies recognized the original his-tagged BmHAXT vaccine antigen.

Levels of Antigen-Specific Antibody Isotypes. Levels of BmHAXT-specific antibody isotypes (IgG1, IgG2a, IgG2b, IgG3, IgE, IgM, and IgA) were determined in the sera samples using an indirect ELISA as described above. Respective isotype-specific biotinylated goat anti-mouse antibodies (Sigma) and streptavidin-HRP (1:20,000) were used as the secondary antibodies. Color was developed with 1-STEP™ Ultra-TMB. The reaction was stopped using 0.16 M H₂SO₄ and optical density was determined at 450 nm in a BioTek Synergy 2 ELISA reader.

The results showed that compared to the control adjuvant group, all antibody isotypes (IgG1, IgG2a, IgG2b, IgG3, IgA, and IgM), except IgE antibodies were elevated in the sera of mice immunized three times with BmHAXT (tag-free), BmHAXT (ΔCys), BmHAXT (GS), and BmHAXT (ΔCys+GS) (FIG. 2). IgE antibody levels were at the background levels in all animals. There were no significant differences in the levels of serum antibody isotypes (except IgG1 antibodies) between the vaccinated group of mice indicating that the modifications (cysteine to serine mutation or addition of GS linker sequence) did not affect the type of immune responses generated in the mice following immunization with the BmHAXT (ΔCys), BmHAXT (GS), or BmHAXT (ΔCys+GS) vaccines. Immunization with BmHAXT (tag-free) resulted in the generation of significantly (p<0.0001) high serum levels of IgG1 antibodies compared to similar values in the mice immunized with BmHAXT (ΔCys), BmHAXT (GS), and BmHAXT (ΔCys+GS). Collectively, these studies demonstrated that immunization with the modifications (cysteine to serine mutation or addition of GS linker sequence) did not affect the immunogenicity of the BmHAXT vaccine. All vaccinated animals showed similar patterns of immune responses.

Challenge Studies. The vaccination studies confirmed that the immunogenicity of the BmHAXT vaccine was not affected by the modifications to the BmHAXT protein. Therefore, it was determined whether the antibodies generated following immunization with the modified BmHAXT vaccine were protective. To determine the vaccine-induced protection, a micropore chamber challenge method as described by Chauhan et al. ((2018) Front. Immunol. 9:1-11) was used. Briefly, 20 infective larvae of B. malayi were placed in a micropore chamber and surgically implanted into the peritoneum of each mouse. Seventy-two hours following implantation, the micropore chambers were recovered from the peritoneal cavity of each mouse. Contents of each chamber were then emptied and total number of larvae recovered were counted. The larvae were then examined under a phase contrast microscope for adherence of cells and for larval death. Larvae that were transparent, straight, and with no movement were counted as dead. Larvae that were active, coiled, and translucent were counted as live.

The results of this analysis showed that significant protection was conferred by serum antibodies from BmHAXT (tag-free), BmHAXT (ΔCys), and BmHAXT (GS) compared to adjuvant control and BmHAXT (ΔCys+GS) group as evidenced by the larval death in all vaccinated animals (FIG. 3). These findings indicated that the vaccine efficacy of BmHAXT was not affected by the cysteine mutation or addition of GS linker in the BmHAXT.

Cross-Reactivity of BmHAXT Preparations. In this experiment, his-tagged BmHAXT protein was separated on a 12% SDS-PAGE gel and the protein was transferred onto a nitrocellulose membrane by western transfer using a semidry blot apparatus. After blocking the non-specific sites on the nitrocellulose membrane with 3% skim milk, separated proteins were probed with (1) anti-penta His monoclonal antibody (ThermoFisher Scientific); (2) sera from a control mouse that were given only adjuvant; (3) sera from a mouse immunized with BmHAXT (tag-free) protein; (4) sera from a mouse immunized with BmHAXT (ΔCys) protein, (5) sera from mice immunized with BmHAXT (GS) protein and BmHAXT (ΔCys+GS) protein. After washing the membrane with PBST, HRP-conjugated chicken anti-mouse IgG antibody (Thermo Fisher Scientific) was added and incubated on a shaking platform for 1 hour at room temperature. After the incubation, the membranes were washed again with PBST and distilled water and color was developed using ECL Western Blotting Substrate (ThermoFisher Scientific). The results showed that the serum antibodies generated following immunization with all the proteins cross-reacted with the original His-tagged BmHAXT vaccine protein, confirming that the modifications to the original BmHAXT did not alter the immune reactivity, immunogenicity and vaccine efficacy of BmHAXT.

### Example 3: Immunogenicity and Vaccine Efficacy of BmHAXT Fusion Protein in Dogs

Immunization Protocol. Six dogs are divided into two groups of three animals per group. Each animal of the first group receives three rounds of 100 μg dose of BmHAXT vaccine plus 40 μg of alum adsorbed GLA-SE (AL019; TLR4 ligand GLA formulated as an oil-in-water emulsion) on days 0, 28 and 56 given i.m. on the left flank region. Each animal of the second group is used as a control and receives three rounds of adjuvant only on days 0, 28 and 56 given i.m. on the left flank region. In addition, at days −1, 0, 28, 56, and 84, blood samples are collected in EDTA tubes from the saphenous vein of each dog prior to immunization. Serum samples are analyzed for antibody titer (IgG, IgG1, IgG2, IgA, IgM and IgE). Peripheral blood mononuclear cells are analyzed for vaccine-induced memory cells and for their cytokine production. Protective antibodies are determined by performing an ADCC assay. All animals are challenged with 50 drug-sensitive D. immitis larvae to determine protection. Vital signs and clinical laboratory parameters (CBC, urinalysis, liver function) are monitored as is injection site reaction (swelling, redness, pain).

It has been shown that the original BmHAXT fusion protein can provide protective antibodies and reduce worm establishment in dogs. See US 2020/0172585. Given that modifications to the original BmHAXT did not alter the immune reactivity, immunogenicity and vaccine efficacy of the original BmHAXT, it is expected that the BmHAXT (ΔCys), BmHAXT (GS), and BmHAXT (ΔCys+GS) fusion proteins will likewise elicit the production of protective antibodies in dogs thereby killing D. immitis infective larvae and significantly reduce worm establishment.

1. A multivalent immunogenic composition comprising a fusion of four or more antigens from one or more filarial nematodes wherein the fusion further comprises
(i) a His tag;
(ii) a linker between two or more of said antigens;
(iii) replacement of one or more cysteine residues in said antigens with serine, or
(iv) any combination of (i), (ii), and (iii), with the proviso that that when the fusion comprises (i) it further comprises one or both of (ii) or (iii).

2. The multivalent immunogenic composition of claim 1, wherein the filarial nematodes are selected from the group consisting of Brugia malayi, Wuchereria bancroft, Onchocerca volvulus, Loa, Brugia timori, Dirofilaria immitis and Dirofilaria repens.

3. The multivalent immunogenic composition of claim 1, wherein the antigens are protein-based, DNA-based, or a combination thereof.

4. The multivalent immunogenic composition of claim 1, wherein the antigens comprise Abundant Larval Transcript, Tetraspanin, Small Heat Shock Protein (HSP) 12.6, and Thioredoxin Peroxidase 2, or fragments thereof.

5. The multivalent immunogenic composition of claim 1, wherein the fusion comprises a GGGSGGGSGGGS (SEQ ID NO:28) linker between each of the antigens.

6. The multivalent immunogenic composition of claim 1, wherein all cysteine residues in the antigens are replaced with serine.

7. The multivalent immunogenic composition of claim 1, wherein the fusion comprises a GGGSGGGSGGGS (SEQ ID NO:28) linker between each of the antigens and all cysteine residues in the antigens are replaced with serine.

8. The multivalent immunogenic composition of claim 1, wherein the antigens are selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8.

9. The multivalent immunogenic composition of claim 1, wherein the fusion is selected from the group consisting of SEQ ID NO:9, SEQ ID NO:10, SEQ ID NO:11, SEQ ID NO:12, SEQ ID NO:13, SEQ ID NO:14, and SEQ ID NO:15.

10. The multivalent immunogenic composition of claim 1, further comprising an adjuvant.

11. A recombinant vector comprising nucleic acids encoding the fusion of four or more antigens from one or more filarial nematode of claim 1.

12. A recombinant host cell comprising the recombinant vector of claim 11.

13. A method for inducing an immune response in a subject comprising administering the multivalent immunogenic composition of claim 1 to a subject thereby inducing an immune response in the subject.

14. The method of claim 13, further comprising administering one or more additional doses of the immunogenic composition to the subject.

15. The method of claim 13, wherein the multivalent immunogenic composition is administered by subcutaneous or intramuscular injection.

16. The method of claim 13, wherein the multivalent immunogenic composition is administered with an adjuvant.

17. A method for immunizing an animal against filariasis or dirofilariasis comprising administering a multivalent immunogenic composition of claim 1 to a subject thereby immunizing the subject against filariasis or dirofilariasis.

18. The method of claim 17, further comprising administering one or more additional doses of the multivalent immunogenic composition to the subject.

19. The method of claim 17, wherein the immunogenic composition is administered by subcutaneous or intramuscular injection.

20. The method of claim 17, wherein the multivalent immunogenic composition is administered with an adjuvant.
